(19) United States
(12) Patent Application Publication
(10) Pub. No.: US 2023/0323466 A1
(43) Pub. Date: Oct. 12, 2023

(54) DETERMINING CANCER RESPONSIVENESS TO TREATMENT

(71) Applicant: Queensland University of Technology, Brisbane (AU)

(72) Inventors: Mark ADAMS, Brisbane (AU); Derek RICHARD, Brisbane (AU); Kenneth O'BYRNE, Brisbane (AU)

(21) Appl. No.: 18/016,021

(22) PCT Filed: Jul. 15, 2021
(86) PCT No.: PCT/AU2021/050759

§ 371 Date: Jan. 13, 2023

(30) Foreign Application Priority Data
Jul. 15, 2020 (AU) 2020902437

(51) Int. Cl. C12Q 1/6886; A61K 33/243; A61K 45/06; A61K 35/00

(52) U.S. Cl. CPC C12Q 1/6886; A61K 33/243; A61K 45/06; A61K 35/00; C12Q 2600/106; C12Q 2600/158

(57) ABSTRACT

The present disclosure relates to cancer and more particularly, methods of treating and/or determining the responsiveness to treatment and/or prognosis of cancers, such as lung cancer and breast cancer.

### CROSS-REFERENCE TO RELATED APPLICATIONS

The present application claims priority from Australian Provisional Patent Application No. 2020902437 filed on 15 Jul. 2020, the contents of which are incorporated herein by reference in their entirety.

### FIELD

THIS APPLICATION relates to cancer. More particularly, this application relates to methods of treating and/or determining the responsiveness to treatment and/or prognosis of cancers, such as lung cancer.

### BACKGROUND

Cancer is a common cause of death worldwide. A wide range of anti-cancer treatments are currently available and can be effective in treating a number of different cancers. Notwithstanding this, typically only a portion of patients treated with anti-cancer agents, such as chemotherapy and molecularly targeted therapies, demonstrate an objective partial response, indicating that a population of these cancer patients unnecessarily receive potentially toxic anti-cancer therapy. Accordingly, complementary diagnostics are needed to identify the cancer patients who will derive most benefit from treatment, such as chemotherapy.

### SUMMARY

The present invention broadly relates to determining expression levels of CDCA3 as a predictive and/or prognostic marker of the response of cancers to treatment. Such treatments may include a Bora-AurA-PLK1 pathway inhibitor, such as a PLK1 inhibitor, and an EGFR inhibitor. The Bora-AurA-PLK1 pathway inhibitor and the EGFR inhibitor may be administered alone or in combination with a chemotherapeutic agent, and more particularly, a platinum-based chemotherapeutic agent, or a tyrosine kinase inhibitor. In a particular form, the cancer is a cancer of the lung, such as NSCLC, or the breast.

In a first aspect, the invention provides a method of predicting the responsiveness of a cancer to treatment with a Bora-AurA-PLK1 pathway inhibitor in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the Bora-AurA-PLK1 pathway inhibitor in the subject.

Suitably, an increased level of the CDCA3 protein or encoding nucleic acid indicates or correlates with increased responsiveness of the cancer to the Bora-AurA-PLK1 pathway inhibitor; and/or a decreased level of CDCA3 protein or encoding nucleic acid indicates or correlates with decreased responsiveness of the cancer to the Bora-AurA-PLK1 pathway inhibitor.

Suitably, the present method is for predicting the responsiveness of the cancer to treatment with the Bora-AurA-PLK1 pathway inhibitor and a further anti-cancer agent. Accordingly, in some examples, an increased level of the CDCA3 protein or encoding nucleic acid indicates or correlates with increased responsiveness of the cancer to the Bora-AurA-PLK1 pathway inhibitor and the further anti-cancer agent; and/or a decreased level of CDCA3 protein or encoding nucleic acid indicates or correlates with decreased responsiveness of the cancer to the Bora-AurA-PLK1 pathway inhibitor and the further anti-cancer agent.

Suitably, the method of the present aspect includes the further step of treating the cancer in the subject. In some examples, wherein an increased level of CDCA3 protein or encoding nucleic acid indicates or correlates with increased responsiveness of the cancer to the Bora-AurA-PLK1 pathway inhibitor and optionally the further anti-cancer agent, the method further includes the step of administering to the subject a therapeutically effective amount of the Bora-AurA-PLK1 pathway inhibitor and optionally the further anti-cancer agent.

In a second aspect, the invention provides a method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of a Bora-AurA-PLK1 pathway inhibitor.

Suitably, the cancer treatment comprises administration of a Bora-AurA-PLK1 pathway inhibitor and a further anti-cancer agent.

In various examples, wherein an increased level of CDCA3 protein or encoding nucleic acid is determined, the method further includes the step of administering to the subject a therapeutically effective amount of the Bora-AurA-PLK1 pathway inhibitor and optionally the further anti-cancer agent.

In a third aspect, the present disclosure relates to a method of treating cancer in a subject, said method including the step of administering a therapeutically effective amount of a Bora-AurA-PLK1 pathway inhibitor to the subject in which a level of CDCA3 protein or encoding nucleic acid has been determined in one or a plurality of cancer cells, tissues or organs of the subject that indicates or correlates with increased responsiveness of the cancer to the Bora-AurA-PLK1 pathway inhibitor.

Suitably, the method further includes administering a therapeutically effective amount of a further anti-cancer agent to the subject. In this regard, the level of CDCA3 protein or encoding nucleic acid can indicate or correlate with increased responsiveness of the cancer to the Bora-AurA-PLK1 pathway inhibitor and the further anti-cancer agent.

In a fourth aspect, the present disclosure provides a method of treating cancer in a subject, said method including the step of administering a therapeutically effective amount of an agent that increases the expression and/or activity of CDCA3 and a Bora-AurA-PLK1 pathway inhibitor to the subject.

Suitably, a decreased level of CDCA3 protein or encoding nucleic acid has been determined in one or a plurality of cancer cells, tissues or organs of the subject. As such, the present method may include the earlier step of determining an expression level of a CDCA3 protein or encoding nucleic acid in the one or plurality of cancer cells, tissues or organs of the subject.

Suitably, the method further includes administering a therapeutically effective amount of a further anti-cancer agent to the subject.

In a fifth aspect, the invention provides a kit for predicting the responsiveness of a cancer to treatment with a Bora-AurA-PLK1 pathway inhibitor in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the Bora-AurA-PLK1 pathway inhibitor.

Suitably, the expression level of the CDCA3 protein or encoding nucleic acid indicates or correlates with increased or decreased responsiveness of the cancer to the Bora-AurA-PLK1 pathway inhibitor and a further anti-cancer agent.

In particular examples, an increased level of CDCA3 protein or encoding nucleic acid indicates or correlates with increased responsiveness of the cancer to the Bora-AurA-PLK1 pathway inhibitor and optionally the further anti-cancer agent; and/or a decreased level of CDCA3 protein or encoding nucleic acid indicates or correlates with decreased responsiveness of the cancer to the Bora-AurA-PLK1 pathway inhibitor and optionally the further anti-cancer agent.

Suitably, the present kit further includes a collection of data comprising correlation data or reference data for correlating the expression level of the CDCA3 protein or encoding nucleic acid and responsiveness of the cancer to the Bora-AurA-PLK1 pathway inhibitor and optionally the further anti-cancer agent.

Suitably, the collection of data or reference data is on a computer-readable medium.

In particular examples, the kit is for use in the method of the aforementioned aspects.

Referring to the aforementioned aspects, the Bora-AurA-PLK1 pathway inhibitor suitably is selected from the group consisting of a bora inhibitor, an Aurora kinase A inhibitor, a PLK1 inhibitor and any combination thereof. In some examples, the Bora-AurA-PLK1 pathway inhibitor is or comprises a PLK1 inhibitor, such as BI2536 and Volasertib (BI6727).

With respect to the above aspects, the further anti-cancer agent suitably is or comprises a chemotherapeutic agent. In some examples, the chemotherapeutic agent is or comprises a platinum-based chemotherapeutic agent. More particularly, the platinum-based chemotherapeutic agent can be selected from the group consisting of cisplatin, carboplatin, oxaliplatin, nedaplatin, triplatin tetranitrate, phenanthriplatin, picoplatin, satraplatin, lipoplatin and any combination thereof. Even more particularly, the platinum-based chemotherapeutic agent can be or comprise cisplatin and/or a derivative thereof.

In other examples of the aforementioned aspects, the further anti-cancer agent is or comprises an inhibitor of a tyrosine kinase, such as EGFR. In this regard, the further anti-cancer agent can be a small molecule inhibitor or an antibody or antibody fragment.

In a sixth aspect, the invention provides a method of predicting the responsiveness of a cancer to treatment with an inhibitor of a tyrosine kinase in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the inhibitor of the tyrosine kinase in the subject.

Suitably, an increased level of the CDCA3 protein or encoding nucleic acid indicates or correlates with increased responsiveness of the cancer to the inhibitor of the tyrosine kinase; and/or a decreased level of CDCA3 protein or encoding nucleic acid indicates or correlates with decreased responsiveness of the cancer to the inhibitor of the tyrosine kinase.

Suitably, the present method is for predicting the responsiveness of the cancer to treatment with the inhibitor of the tyrosine kinase and a further anti-cancer agent. To this end and in particular examples, an increased level of the CDCA3 protein or encoding nucleic acid indicates or correlates with increased responsiveness of the cancer to the inhibitor of the tyrosine kinase and optionally the further anti-cancer agent; and/or a decreased level of CDCA3 protein or encoding nucleic acid indicates or correlates with decreased responsiveness of the cancer to the inhibitor of the tyrosine kinase and optionally the further anti-cancer agent.

Suitably, the method of the present aspect includes the further step of treating the cancer in the subject. In various examples, wherein an increased level of CDCA3 protein or encoding nucleic acid indicates or correlates with increased responsiveness of the cancer to the inhibitor of the tyrosine kinase and optionally the further anti-cancer agent, the method further includes the step of administering to the subject a therapeutically effective amount of the inhibitor of the tyrosine kinase and optionally the further anti-cancer agent.

In a seventh aspect, the present disclosure provides a method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of an inhibitor of a tyrosine kinase.

Suitably, the cancer treatment comprises administration of the inhibitor of the tyrosine kinase and a further anti-cancer agent.

In various examples, wherein an increased level of CDCA3 protein or encoding nucleic acid is determined, the method further includes the step of administering to the subject a therapeutically effective amount of the inhibitor of the tyrosine kinase and optionally the further anti-cancer agent.

In an eighth aspect, the present disclosure relates to a method of treating cancer in a subject, said method including the step of administering a therapeutically effective amount of an inhibitor of a tyrosine kinase to the subject in which a level of CDCA3 protein or encoding nucleic acid has been determined in one or a plurality of cancer cells, tissues or organs of the subject that indicates or correlates with increased responsiveness of the cancer to the inhibitor of the tyrosine kinase.

Suitably, the method further includes administering a therapeutically effective amount of a further anti-cancer agent to the subject. In this regard, the level of CDCA3 protein or encoding nucleic acid can indicate or correlate with increased responsiveness of the cancer to the inhibitor of the tyrosine kinase and the further anti-cancer agent.

In a ninth aspect, the present disclosure provides a method of treating cancer in a subject, said method including the step of administering a therapeutically effective amount of an agent that increases the expression and/or activity of CDCA3 and an inhibitor of a tyrosine kinase to the subject.

Suitably, a decreased level of CDCA3 protein or encoding nucleic acid has been determined in one or a plurality of cancer cells, tissues or organs of the subject. As such, the present method may include the earlier step of determining an expression level of a CDCA3 protein or encoding nucleic acid in the one or plurality of cancer cells, tissues or organs of the subject.

Suitably, the method further includes administering a therapeutically effective amount of a further anti-cancer agent to the subject.

In a tenth aspect, the present disclosure provides a kit for predicting the responsiveness of a cancer to treatment with an inhibitor of a tyrosine kinase in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the inhibitor of the tyrosine kinase.

Suitably, the expression level of the CDCA3 protein or encoding nucleic acid indicates or correlates with increased or decreased responsiveness of the cancer to the inhibitor of the tyrosine kinase and a further anti-cancer agent.

In particular examples, an increased level of CDCA3 protein or encoding nucleic acid indicates or correlates with increased responsiveness of the cancer to the inhibitor of the tyrosine kinase and optionally the further anti-cancer agent; and/or a decreased level of CDCA3 protein or encoding nucleic acid indicates or correlates with decreased responsiveness of the cancer to the inhibitor of the tyrosine kinase and optionally the further anti-cancer agent.

Suitably, the present kit further includes a collection of data comprising correlation data or reference data for correlating the expression level of the CDCA3 protein or encoding nucleic acid and responsiveness of the cancer to the inhibitor of the tyrosine kinase and optionally the further anti-cancer agent.

Suitably, the collection of data or reference data is on a computer-readable medium.

In particular examples, the kit is for use in the method of the sixth, seventh, eighth and ninth aspects.

Referring to the five aforementioned aspects, the inhibitor of the tyrosine kinase suitably is an EGFR inhibitor, such as erlotinib, afatinib and osimertinib.

With respect to the sixth, seventh, eighth, ninth and tenth aspects, the further anti-cancer agent suitably is or comprises a chemotherapeutic agent. In some examples, the chemotherapeutic agent is or comprises a platinum-based chemotherapeutic agent. More particularly, the platinum-based chemotherapeutic agent can be selected from the group consisting of cisplatin, carboplatin, oxaliplatin, nedaplatin, triplatin tetranitrate, phenanthriplatin, picoplatin, satraplatin, lipoplatin and any combination thereof. Even more particularly, the platinum-based chemotherapeutic agent can be or comprise cisplatin and/or a derivative thereof.

In other examples of the sixth to tenth aspects, the further anti-cancer agent can be or comprise a Bora-AurA-PLK1 pathway inhibitor. Suitably, the Bora-AurA-PLK1 pathway inhibitor is selected from the group consisting of a bora inhibitor, an Aurora kinase A inhibitor, a PLK1 inhibitor and any combination thereof. In some examples, the Bora-AurA-PLK1 pathway inhibitor is or comprises a PLK1 inhibitor, such as BI2536 and Volasertib (BI6727).

In some examples of the aforementioned aspects, the method or kit is or comprises a companion diagnostic.

Suitably, the cancer of the aforementioned aspects is or comprises a lung cancer. In some examples, the lung cancer can be selected from squamous cell carcinoma, adenocarcinoma, large cell carcinoma, small cell carcinoma and mesothelioma. In alternative examples, the cancer of the aforementioned aspects is or comprises a breast cancer, such as triple negative breast cancer.

Suitably, the subject of the above aspects is a mammal, and in particular examples a human.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

The indefinite articles ‘a’ and ‘an’ are used herein to refer to or encompass singular or plural elements or features and should not be taken as meaning or defining “one” or a “single” element or feature. For example, “a” cell includes one cell, one or more cells and a plurality of cells.

### BRIEF DESCRIPTION OF THE FIGURES

FIG. 1. Schematic representing an example of a method of the present disclosure to identify and enhance chemotherapy sensitivity in NSCLC patients using CDCA3 expression as a diagnostic marker.

FIG. 2. (A) Undamaged cells depleted of CDCA3 progress to mitosis slower than control cells or cells complemented with siRNA resistant CDCA3. (B) Cisplatin treated cells and depleted of CDCA3 are unable to recover from cell cycle arrest. (C) Cells treated with Cisplatin and then a PLK1 inhibitor are unable to recover from cell cycle arrest.

FIG. 3. (A) GST-CDCA3 pulldown of PLK1 and Aurora A but not WEE1 from G2 synchronised cells. (B) CDCA3 immunoprecipitates with PLK1 and Aurora A. (C) PLK1 immunoprecipitates with CDCA3 from lysates of G2 synchronised cells (G2=˜7-10 h) but not lysates collected from cells synchronised at the G1/S cell cycle boundary or in prometaphase (mitosis). (D) CDCA3 is capable of immunoprecipitating with PLK1 in the absence of Aurora A. (E) CDCA3 and PLK1 immunoprecipitate in unperturbed G2 cells and irrespective of activation of the G2 checkpoint with ionising radiation (IR).

FIG. 4. (A) Schematic of the FRET-based biosensor. FRET occurs in the basal state. Phosphorylation of the designated sequence (yellow) leads to binding with the FHA2 phospho-binding domain resulting in a conformation change and separation of the CFP and YFP fluorophores (low FRET) [33]. (B) CDCA3 depletion reduces PLK1 activity in late G2. Timecourse of the percentage change in FRET ratio of the PLK1-biosensor in G2 synchronised control siRNA (upper) or CDCA3 depleted (lower) cells and in control cells treated with the PLK1 inhibitor BI 2536. (C) CDCA3 depletion prevents timely PLK1 activation (phospho-PLK1 T210) and the binding of PLK1 with Aurora A (low band), the kinase required to activate PLK1. (D) CDCA3 depletion and PLK1 inhibition (in a dose responsive manner) reduces mitotic index, measured by H3S10ph, in cells ectopically expressing wildtype full-length PLK1. Ectopic expression of a constitutively active PLK1 mutant (T210D) increases the mitotic index in control and CDCA3 depleted cells, yet remains sensitive to PLK1 inhibition.

FIG. 5. (A) PLK1 schematic listing kinase domain and polo-box domain (PBD). (B) CDCA3 immunoprecipitates with the PBD of PLK1 irrespective of the phospho-binding mutant (H538 and K540 each mutated to alanine residues). (C) CDCA3 is phosphorylated by CDK1-cyclin B1 causing a large molecular weight shift measured by SDS-PAGE. (D) Mutating each of the five CDK1-cyclin B1 phosphorylation sites within CDCA3 enhances binding to PLK1. (E) Inhibition of CDK1 with RO3306 enhances immunoprecipitation of CDCA3 with the PBD domain of PLK1. (F) Unmodified recombinant CDCA3 binds to recombinant Aurora A and PLK1. CDCA3 pre-phosphorylated by CDK1-cyclin B1 binds markedly less to PLK1 than unmodified CDCA3. (G) Recombinant unmodified CDCA3 but not phosphorylated CDCA3 enhances the in vitro activation and phosphorylation of PLK1 by Aurora A.

FIG. 6. (A-F) Scatter plots showing linear regression analysis of The Cancer genome Atlas (TCGA) RNAseq datasets assessing the correlation between CDCA3 levels and measures of genome instability (A-D) and predicted chemotherapy sensitivity (E-F) in NSCLC. R and P values determined according to Spearman's rank correlation. (A-B) Correlation with gene expression signature reflective of a homologous recombination deficiency (HRD) score in adenocarcinoma (ADC, A) and squamous cell carcinoma (SqCC, B). (C-D) Correlation in ADC (C) and SqCC (D) between CD CA3 levels and HRD score calculated by the unweighted sum of three genomic scars, telomeric allelic imbalances, large scale genomic transitions and loss of heterozygosity. (E-F) Correlation with gene expression signature reflective of a pharmacogenomic predictor of pathologic complete response (pCR) to preoperative chemotherapy in ADC (E) and SqCC (F). (G) Scatter plot showing linear regression analysis assessing correlation between CDCA3 protein levels, determined by previous western blot analysis, and in vitro cisplatin sensitivity which is represented by IC₅₀ values. Cisplatin IC₅₀ values calculated by plotting NSCLC cell viability for each of the escalating cisplatin doses (see Extended data FIG. 1). R and P values determined according to Spearman's rank correlation. (H) Beeswarm plots showing the foci count per nucleus of FANCI immunofluorescence microscopy for five NSCLC cell lines grouped by high or low CDCA3 protein levels (determined in (G)). Cells endogenously expressing high versus low CDCA3 levels were untreated, cisplatin treated for 12 h (cisplatin) or cultured in fresh growth medium for 8 h following cisplatin treatment (recovery). Data points represent an average of FANCI foci/nuclei per field of view from a minimum of 800 nuclei (n=13-16 fields). Blue lines indicate median values. Dotted black lines highlight change in foci count following recovery. Percent recovery calculated by difference between 100% and ratio of recovery/cisplatin expressed as a percentage.

FIG. 7. CDCA3 regulates NSCLC cell proliferation by enabling efficient cell cycle progression in cell lines demonstrating elevated CDCA3 transcript & protein in vitro.

FIG. 8. CDCA3-depletion reduces cisplatin IC50 values ˜2-4 fold versus control NSCLC cells but not non-tumorigenic lung epithelial HBEC cells, indicating increased sensitivity to cisplatin in NSCLC in vitro.

FIG. 9. (A) PLK1 inhibitors are more potent (measured by IC50 value) in NSCLC cell lines expressing high levels of CDCA3 versus lower endogenous CDCA3 expression levels. (B) Combination of cisplatin with BI 2536 is additive in control cells but not in CDCA3 depleted cells [Theoretical additivity=% drug A+% drug B(100−% drug A)/100].

FIG. 10. (A) Kaplan-Meier analysis of overall survival of 1402 breast cancer cases comparing high versus low CDCA3 transcript levels split by median expression. Patients with elevated CDCA3 transcript levels have a poorer outcome than patients with lower levels of CDCA3. (B) CDCA3 protein is significantly elevated in triple negative breast cancer cell lines versus breast cancer cell lines positive for either estrogen receptor (ER), progesterone receptor (PR) or human epidermal growth factor receptor-2 (HER2). (D) Combination of cisplatin with BI 2536 is additive in triple negative breast cancer control MDA-MB-468 cells but not in CDCA3 depleted cells [Theoretical additivity=% drug A+% drug B(100−% drug A)/100].

FIG. 11. CDCA3 levels are elevated in EGFR^{mut}NSCLC cell lines. Stimulation of receptor tyrosine kinase (RTK) signalling in EGFR wildtype cells induces the upregulation of CDCA3 protein.

FIG. 12. Tyrosine kinase inhibitors suppress CDCA3 levels in EGFR^{mut} NSCLC cell lines. CDCA3 levels are suppressed by TKIs in CDCA3^{high} exon 19 deleted EGFR^{mut} (HCC827) and only by third generation TKI in T790M EGFR^{mut}. CDCA3 levels are unaffected by TKI in CDCA3^{low} EGFR^{mut}.

FIG. 13. First, second and third generation tyrosine kinase inhibitors are less potent in CDCA3^{low} EGFR^{mut}NSCLC cell lines.

FIG. 14. PLK1 inhibition enhances TKI sensitivity in CDCA3^{high} EGFR^{mut}NSCLC cell lines.

FIG. 15. CDCA3 correlates with sensitivity to EGFR TKIs

FIG. 16. Upregulating CDCA3 protein levels in models of acquired EGFR TKI resistance enhances TKI sensitivity

### DETAILED DESCRIPTION

The present disclosure is at least partly predicated on the surprising discovery that CDCA3 is a predictive biomarker of response or resistance to therapy with a PLK1 inhibitor alone or in combination with platinum-based chemotherapy in cancer. To this end, CDCA3 levels may be utilized to determine whether combining a PLK1 inhibitor with the platinum-based chemotherapy can be effective in overcoming resistance to the latter. The present disclosure is also at least partly predicated on the surprising finding that CDCA3 is a predictive biomarker of response or resistance to therapy with an EGFR inhibitor alone or in combination with a PLK1 inhibitor in cancer.

In one particular aspect, the present disclosure provides a method of predicting the responsiveness of a cancer to treatment with a Bora-AurA-PLK1 pathway inhibitor in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the Bora-AurA-PLK1 pathway inhibitor in the subject.

Suitably, the present method is for predicting the responsiveness of the cancer to treatment with the Bora-AurA-PLK1 pathway inhibitor and a further anti-cancer agent. Accordingly in one form, the present disclosure provides a method of predicting the responsiveness of a cancer to treatment with a Bora-AurA-PLK1 pathway inhibitor and optionally a further anti-cancer agent in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the Bora-AurA-PLK1 pathway inhibitor and optionally the further anti-cancer agent in the subject.

It would be understood by the skilled artisan that the CDCA3 gene comprises a nucleotide sequence that encodes the protein Cell Division Cycle Associated 3. Other names for CDCA3 may include Trigger of Mitotic Entry Protein 1, Gene-Rich Cluster Protein C8, TOME-1, GRCC and C8. Non-limiting examples of Accession Numbers referencing the nucleotide sequence of the CDCA3 gene, or its encoded protein, as are well understood in the art, in humans include NM_031299.6, NM_001331019.1, NM_001297602.2 NM_001297603.2, NM_001297604.2, NP_001284533.1, NP_001317948.1, NP_001284531.1, NP_001284532.1, NP_112589.1, albeit without limitation thereto. As generally used herein, “CDCA3” may refer to a CDCA3 nucleic acid or encoded protein, unless otherwise specified.

For the purposes of this disclosure, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein a “gene” is a nucleic acid which is a structural, genetic unit of a genome that may include one or more amino acid-encoding nucleotide sequences and one or more non-coding nucleotide sequences inclusive of promoters and other 5′ untranslated sequences, introns, polyadenylation sequences and other 3′ untranslated sequences, although without limitation thereto. In most cellular organisms a gene is a nucleic acid that comprises double-stranded DNA.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Also included are, “variant” nucleic acids that include nucleic acids that comprise nucleotide sequences of naturally occurring (e.g., allelic) variants and orthologs (e.g., from a different species) of CDCA3. Suitably, nucleic acid variants share at least 70% or 75%, particularly at least 80% or 85% or more particularly at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. A non-limiting example is an amplification product or a primer or probe. In particular examples, a nucleic acid fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700 and 800, 900, 1000, 1500 and 2000 contiguous nucleotides of said nucleic acid.

As used herein, a “polynucleotide” is a nucleic acid having eighty (80) or more contiguous nucleotides, while an “oligonucleotide” has less than eighty (80) contiguous nucleotides. A “probe” may be a single or double-stranded oligonucleotide or polynucleotide, suitably labelled for the purpose of detecting complementary sequences in Northern or Southern blotting, for example. A “primer” is usually a single-stranded oligonucleotide, suitably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “template” nucleic acid is a nucleic acid subjected to nucleic acid amplification.

By “protein” is meant an amino acid polymer. The amino acids may be natural or non-natural amino acids, D- or L- amino acids as are well understood in the art. As would be appreciated by the skilled person, the term “protein” also includes within its scope phosphorylated forms of a protein (i.e., a phosphoprotein) and/or glycosylated forms of a protein (i.e. a glycoprotein). A “peptide” is a protein having no more than fifty (50) amino acids. A “polypeptide” is a protein having more than fifty (50) amino acids.

Also provided are protein “variants” such as naturally occurring (e.g. allelic variants) and orthologs of CDCA3. Suitably, protein variants share at least 70% or 75%, particularly at least 80% or 85% or more particularly at least 90%, 91%, 92%, 93%, 94%, 95%, 96%, 97%, 98% or 99% sequence identity with an amino acid sequence of CDCA3 disclosed herein or known in the art.

Also provided are protein fragments, inclusive of peptide fragments that comprise less than 100% of an entire amino acid sequence. In particular examples, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, and 260 contiguous amino acids of said protein.

As generally used herein, the terms “cancer”,“tumour”,“malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

With respect to the present and hereinafter described aspects, the cancer suitably is, or comprises, a lung cancer. To this end, it would be apparent that lung cancer may include any aggressive lung cancers and cancer subtypes known in the art, such as non-small cell carcinoma or non-small cell lung cancer (i.e., squamous cell carcinoma, adenocarcinoma and large cell carcinoma), small cell carcinoma and mesothelioma. In particular examples, the lung cancer is or comprises non-small cell lung cancer (NSCLC). As such, the lung cancer can be squamous cell carcinoma, adenocarcinoma or large cell carcinoma. In certain examples, the lung cancer is a squamous cell carcinoma or an adenocarcinoma. In particular examples, the lung cancer is a squamous cell carcinoma. In some examples, the lung cancer is an adenocarcinoma. In other examples, the lung cancer is a large cell carcinoma.

Again, referring to the aspects of the present disclosure described herein, the cancer suitably is, or comprises, a breast cancer. The skilled person will appreciate that the breast cancer may include any aggressive breast cancers and cancer subtypes known in the art, such as triple negative breast cancer, grade 2 breast cancer, grade 3 breast cancer, lymph node positive (LN+) breast cancer, HER2 positive (HER2+) breast cancer, PR negative (PR⁻) breast cancer, PR positive (PR⁺) breast cancer, ER negative (ER⁻) breast cancer and ER positive (ER+) breast cancer. In various examples, the breast cancer is or comprises triple negative breast cancer.

Suitably, the Bora-AurA-PLK1 pathway inhibitor is or comprises one or more of a Bora inhibitor, an Aurora kinase A inhibitor and a PLK1 inhibitor. In certain examples, the Bora-AurA-PLK1 pathway inhibitor is or comprises a PLK1 inhibitor.

The cancer described herein can include an EGFR mutation, such as an activating EGFR mutation or an EGFR gene amplification, and/or is at least partly associated with increased EGFR activity. In this regard, the cancer may be considered to be an EGFR mutation-positive cancer.

Exemplary EGFR mutations, such as EGFR activating mutations that may be associated with cancer include point mutations, deletion mutations, insertion mutations, inversions or gene amplifications that lead to an increase in at least one biological activity of EGFR, such as elevated tyrosine kinase activity, formation of receptor homodimers and heterodimers, enhanced ligand binding etc. Mutations can be located in any portion of an EGFR gene or regulatory region associated with an EGFR gene and include mutations in exon 18, 19, 20 or 21. Other examples of EGFR activating mutations are known in the art (see e.g., U.S. Pat. Publ. No. US2005/0272083).

In some examples, the EGFR mutation is or comprises E709K, L718Q, L718V, G719A, G719X, G724X, G724S, I744T, E746K, L747S, E749Q, A750P, A755V, V765M, C775Y, T790M, L792H, L792V, G796S, G796R, G796C, C797S, T8541, L858P, L858R, L861X, delE746-A750, delE746_T751InsKV, delE746_A750InsHS, delE746_T751InsFPT, delE746_T751InsL, delE746_S752InsIP, delE746_P753InsMS, delE746_T751InsA, delE746_T751InsAPT, delE746_T751InsVA, delE746_S752InsV, delE746_P753InsVS, delE746_K754InsGG, delE746_E749, delE746_E749InsP, delL747_E749, delL747_A750InsP, delL747_T751InsP, delL747_T751InsN, delL747_S752InsPT, delL747_P753InsNS, delL747_S752InsPI, delL747_S752, delL747_P753InsS, delL747_K754, delL747_T751InsS, delL747_T751, delL747_P753InsS, delA750_I759InsPT, delT751_I759InsT, delS752_1759, delT751_I759InsN, delT751_D761InsNLY, delS752_I759, de1R748-P753, delL747-P753insS, delL747-T751, M766_A767InsA, S768_V769InsSVA, P772_H773InsNS, D761_E762InsX, A763_Y764InsX, Y764_Y765 InsX, M766_A767InsX, A767_V768 InsX, S768_V769 InsX, V769_D770 InsX, D770_N771 InsX, N771_P772 InsX, P772_H773 InsX, H773_V774 InsX, V774_C775 InsX, one or more deletions in EGFR exon 20, or one or more insertions in EGFR exon 20, one or more deletions in EGFR exon 19, or one or more insertions in EGFR exon 19, or any combination thereof, wherein X refers to any of the naturally occurring amino acids and can be one to seven amino acids long. The nomenclature of the mutations is well-known. In particular examples, the EGFR mutation is or comprises one or more deletions in exon 19, L858R, T790M and any combination thereof.

As will be appreciated, the term “PLK1 inhibitor” refers to a drug for inhibiting polo-like kinase 1. It will be apparent to the skilled artisan that when a PLK1 inhibitor is administered to a subject the PLK1 activity within the subject is altered, and more particularly reduced. A drug able to decrease the expression level of PLK1 expression is also considered a PLK1 inhibitor. In various examples, a prodrug of a PLK1 inhibitor is administered to a subject that is converted to the compound in vivo where it inhibits PLK1.

The PLK1 inhibitor may be any type of compound. For example, the compound may be a small organic molecule or a biological compound such as an antibody or an enzyme. To this end, a person skilled in the art can determine whether a compound is capable of inhibiting PLK1 activity and/or expression by any means known in the art. Exemplary assays for evaluating PLK1 activity and/or inhibition thereof include, for example, dot blots and kinase assays that measure the direct kinase activity of PLK1 (e.g., measures ADP formed from a kinase reaction). It will be appreciated that the PLK1 inhibitor may be any known in the art, such as volasertib (BI6727), rigosertib (ON01910), GSK461364A, ZK-thiazolidinone, cyclapolin 9, TKM-080301, GW843682, purpurogallin, poloxin, poloxin-2, R03280, NMS-P937 (also referred to as NMS1286937), MLN0905, BI 2536, SBE 13 hydrochloride, TAK960 hydrochloride and any combination thereof. In particular examples, the PLK1 inhibitor is or comprises BI2536. In some examples, the PLK1 inhibitor is or comprises BI6727 (volasertib).

In particular examples, the further anti-cancer agent referred to herein is or comprises a chemotherapeutic agent. Accordingly, in certain examples, the treatments described herein include a Bora-AurA-PLK1 pathway inhibitor and a chemotherapeutic agent. In particular, examples the treatments described herein include a PLK1 inhibitor and a chemotherapeutic agent.

As generally used herein, the term “chemotherapy” or “chemotherapeutic agent” broadly refers to a treatment or agent with a cytostatic or cytotoxic agent (i.e., a compound) to reduce or eliminate the growth or proliferation of undesirable cells, such as cancer cells. Accordingly, the terms can refer to a cytotoxic or cytostatic agent used to treat a proliferative disorder, for example cancer. The cytotoxic effect of the agent can be, but is not required to be, the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Exemplary chemotherapeutic agents include, but are not limited to, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin, oxaliplatin, nedaplatin, triplatin tetranitrate, phenanthriplatin, picoplatin, satraplatin and lipoplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

In various examples, the chemotherapeutic agent is a platinum-based chemotherapeutic agent. The terms “platinum-based chemotherapy” and “platinum-based chemotherapeutic agent” as used interchangeably herein refer to a molecule or a composition comprising a molecule containing a coordination complex comprising the chemical element platinum and is useful as a chemotherapy drug. Platinum-based chemotherapy generally acts by inhibiting DNA synthesis and possesses some alkylating activity. Examples of platinum-based chemotherapy drugs include cisplatin, carboplatin, oxaliplatin, nedaplatin, triplatin tetranitrate, phenanthriplatin, picoplatin, satraplatin and lipoplatin. The platinum-based chemotherapy drug may be administered as a monotherapy, or in combination with other anti-cancer agents (e.g., a PLK1 inhibitor and/or an EGFR inhibitor), or as prodrugs, or together with local therapies such as surgery and radiation, or as adjuvant or neoadjuvant chemotherapy, or as part of a multimodal approach to the treatment of neoplastic disease. In particular examples, the chemotherapeutic agent is cisplatin and/or a derivative thereof.

In some examples, the further anti-cancer agent described herein is or comprises an inhibitor of a tyrosine kinase. The term “tyrosine kinase” refers to enzymes which are capable of transferring a phosphate group from ATP to a tyrosine residue in a protein. Phosphorylation of proteins by tyrosine kinases is an important mechanism in signal transduction for regulation of enzyme activity and cellular events such as cell survival or proliferation. Non-limiting examples of tyrosine kinases include receptor tyrosine kinases such as EGFR (Epidermal growth factor receptor e.g., EGFR/HER1/ErbB1, HER2/Neu/ErbB2, HER3/ErbB3, HER4/ErbB4), INSR (insulin receptor), IGF-IR, IGF-II1R, IRR (insulin receptor-related receptor), PDGFR (e.g., PDGFRA, PDGFRB), c-KIT/SCFR, VEGFR-1/FLT-1, VEGFR-2/FLK-1/KDR, VEGFR-3/FLT-4, FLT-3/FLK-2, CSF-1R, FGFR 1-4, CCK4, TRK A-C, MET, RON, EPHA 1-8, EPHB 1-6, AXL, MER, TYRO3, TIE, TEK, RYK, DDR 1-2, RET, c-ROS, LTK (leukocyte tyrosine kinase), ALK (anaplastic lymphoma kinase), ROR 1-2, MUSK, AATYK 1-3, and RTK 106; and non-receptor tyrosine kinases such as BCR-ABL, Src, Frk, Btk, Csk, Abl, Zap70, Fes/Fps, Fak, Jak, Ack, and LIMK.

The terms “inhibitor of a tyrosine kinase” and “tyrosine kinase inhibitor” are used interchangeably herein and refer to the ability of a compound, such as a small molecule or antibody or antibody fragment, to alter the function of tyrosine kinases. An inhibitor may block or reduce the activity of tyrosine kinases by forming a reversible or irreversible covalent bond between the inhibitor and a tyrosine kinase or a ligand thereof or through formation of a noncovalently bound complex. Such inhibition may be manifest only in particular cell types or may be contingent on a particular biological event. The term “inhibit” or “inhibition” also refers to altering the function of tyrosine kinases by decreasing the probability that a complex forms between a tyrosine kinase and a natural substrate. It will be appreciated that such inhibition of tyrosine kinases may be assessed by any method known in the art. Exemplary methods are described in WO2005/012294; WO2008/064274; WO2006/078846; Weinblatt et al., Arthritis Rheum., 2008, 58(11), 3309-3318; Cha et al., J. Pharmacol. Exp. Ther., 2006, 317(2), 571-578; Bajhat et al., Arthritis & Rheumatism, 2008, 58(5), 1433-1444; and Brasselmann et al., J. Pharmacol. Exp. Ther., 2006, 319(3), 998-1008.

Exemplary tyrosine kinase inhibitors include, but are not limited to, erlotinib (Tarceva); afatinib (Gilotrif), osimertinib (Tagrisso), gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; AMG888; canertinib; BMS-599626 (AC-480); neratinib; KRN-633; CEP-11981; AZM-475271; CP-724714; TAK-165; CP-547632; bosutinib; lestaurtinib; tandutinib; midostaurin; enzastaurin; AEE-788; axitinib; motasenib; OSI-930; cediranib, KRN-951, dovitinib, seliciclib, SNS-032, PD-0332991, MKC-I (Ro-317453; R-440), ABT-869, brivanib (BMS-582664), SU-14813, telatinib, SU-6668, (TSU-68), L-21649, MLN-8054, AEW-541, and PD-0325901.

In particular examples, the further anti-cancer agent is or comprises an EGFR inhibitor, such as those provided herein. Accordingly, in particular examples, the treatment described herein includes a Bora-AurA-PLK1 pathway inhibitor and an EGFR inhibitor. In various examples, the treatment described herein includes a PLK1 inhibitor and an EGFR inhibitor.

The term “epidermal growth factor receptor” or “EGFR” as used herein refers to a transmembrane protein that is a receptor for members of the epidermal growth factor family (EGF family) of extracellular protein ligands. It refers to a tyrosine kinase which regulates signalling pathways and growth and survival of cells and which shows affinity for the EGF molecule. The ErbB family of receptors consists of four closely related subtypes: ErbB1 (epidermal growth factor receptor; EGFR), ErbB2 (HER2/neu), ErbB3 (HER3), and ErbB4 (HER4) and variants thereof (e.g., a deletion mutant EGFR as in Humphrey et al. (Proc. Natl. Acad. Sci. USA, 1990, 87:4207-4211). Binding of an EGF ligand activates the EGFR (e.g., resulting in activation of intracellular mitogenic signalling and autophosphorylation of EGFR). One of skill in the art will understand that other ligands, in addition to EGF, can bind to and activate the EGFR. Examples of such ligands include, but are not limited to, amphiregulin, epiregulin, TGF-α, betacellulin (BTC), and heparin-binding EGF (HB-EGF).

As used herein, the term “EGFR inhibitor” refers to compounds that bind to or otherwise interact directly with EGFR (or any of its sequence variants, deletion or insertion mutants as are known in the art) and prevent or reduce its signalling activity, and is alternatively referred to as an “EGFR antagonist”. Examples of such agents include antibodies and small molecules that bind to EGFR.

Exemplary EGFR inhibitors include the anti-EGFR antibodies: cetuximab (Erbitux®), panitumumab (Vectibix®), matuzumab, nimotuzumab; and small molecule EGFR inhibitors: Tarceva® (erlotinib), IRESSA (gefitinib), osimertinib, EKB-569 (pelitinib, irreversible EGFR TKI), pan-ErbB and other receptor tyrosine kinase inhibitors, lapatinib (EGFR and HER2 inhibitor), pelitinib (EGFR and HER2 inhibitor), vandetanib (ZD6474, ZACTIMA™, EGFR, VEGFR2 and RET TKI), PF00299804 (dacomitinib, irreversible pan-ErbB TKI), CI-1033 (irreversible pan-erbB TKI), afatinib (BIBW2992, irreversible pan-ErbB TKI), AV-412 (dual EGFR and ErbB2 inhibitor), EXEL-7647 (EGFR, ErbB2, GEVGR and EphB4 inhibitor), CO-1686 (irreversible mutant-selective EGFR TKI), AZD9291 (irreversible mutant-selective EGFR TKI), and HKI-272 (neratinib, irreversible EGFR/ErbB2 inhibitor). In some examples, the EGFR inhibitor is or comprises erlotinib. In particular examples, the EGFR inhibitor is osimertinib.

In other examples, the further anti-cancer agent described herein is or comprises an anti-angiogenic agent, such as a VEGF/VEGFR inhibitor. Accordingly, in particular examples, the treatment described herein includes a Bora-AurA-PLK1 pathway inhibitor and a VEGF/VEGFR inhibitor. In various examples, the treatment described herein includes a PLK1 inhibitor and a VEGF/VEGFR inhibitor. Exemplary VEGF/VEGFR inhibitors include, but are not limited to, bevacizumab (Avastin); sorafenib (Nexavar); sunitinib (Sutent); ranibizumab; pegaptanib; or vandetinib.

In view of the above, the cancer described herein may include a VEGF or VEGFR mutation, such as an activating VEGF/VEGFR mutation or a VEGF/VEGFR gene amplification, and/or is at least partly associated with increased VEGF/VEGFR activity. In this regard, the cancer may be considered to be a VEGF and/or VEGFR mutation-positive cancer.

In examples relating to antibody inhibitors, such as PLK1 inhibitors and inhibitors of a tyrosine kinase, the antibody may be polyclonal or monoclonal, native or recombinant. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

As would be understood by the skilled person, the expression level of a CDCA3 nucleic acid or encoded protein may be relatively (i) higher, increased or greater; or (ii) lower, decreased or reduced when compared to an expression level in a control or reference sample, or to a threshold expression level. In various examples, an expression level may be classified as higher, increased or greater if it exceeds a mean and/or median expression level of a reference population. In some examples, an expression level may be classified as lower, decreased or reduced if it is less than the mean and/or median expression level of the reference population. In this regard, a reference population may be a group of subjects who have the same cancer type, subgroup, stage and/or grade as said mammal for which the expression level is determined.

Terms such as “higher”, “increased” and “greater” as used herein refer to an elevated amount or level of a CDCA3 nucleic acid or protein, such as in a biological sample, when compared to a control or reference level or amount. The expression level of the CDCA3 nucleic acid or protein may be relative or absolute (i.e., relatively or absolutely higher, increased or greater). In some examples, the expression of the CDCA3 nucleic acid or protein is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of expression of a CDCA3 nucleic acid or protein in a control or reference level or amount.

The terms, “lower”, “reduced” and “decreased”, as used herein refer to a lower amount or level of the CDCA3 nucleic acid or protein, such as in a biological sample, when compared to a control or reference level or amount. The expression level of the CDCA3 nucleic acid or protein may be relative or absolute (i.e., relatively or absolutely lower, reduced or decreased). In some examples, the expression of the CDCA3 nucleic acid or protein is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of expression of the CDCA3 nucleic acid or protein in a control or reference level or amount.

The term “control sample” typically refers to a biological sample from a (healthy) non-diseased individual not having cancer. In some examples, the control sample may be from a subject known to be free of cancer. Alternatively, the control sample may be from a subject in remission from cancer. The control sample may be a pooled, average or an individual sample. An internal control is a marker from the same biological sample being tested.

As used herein, an expression level may be an absolute or relative amount of an expressed nucleic acid or protein. Accordingly, in some examples, the expression level of the CDCA3 gene and/or a product thereof is compared to a control level of expression, such as the level of gene and/or protein expression of one or a plurality of “housekeeping” genes in one or more cancer cells, tissues or organs of the mammal.

In further examples, the expression level of the CDCA3 nucleic acid or encoded protein is compared to a threshold level of expression, such as a level of gene and/or protein expression in non-cancerous tissue or cells. A threshold level of expression is generally a quantified level of expression of CDCA3. Typically, an expression level of CDCA3 in a sample that exceeds or falls below the threshold level of expression is predictive of a particular disease state or outcome, such as resistance or responsiveness of the subject's cancer to the Bora-AurA-PLK1 pathway inhibitor or an inhibitor of a tyrosine kinase and optionally the further anti-cancer agent (e.g., a chemotherapeutic agent). The nature and numerical value (if any) of the threshold level of expression will typically vary based on the method chosen to determine the expression of the one or more genes, or products thereof, used in determining, for example, a prognosis and/or a response to the Bora-AurA-PLK1 pathway inhibitor or an inhibitor of a tyrosine kinase and optionally the further anti-cancer agent (e.g., the chemotherapeutic agent), in the mammal.

A person of skill in the art would be capable of determining the threshold level of CDCA3 nucleic acid or protein expression in a sample that may be used in determining, for example, a prognosis and/or a response to the Bora-AurA-PLK1 pathway inhibitor or an inhibitor of a tyrosine kinase and optionally the further anti-cancer agent, using any method of measuring gene or protein expression known in the art, such as those described herein. In various examples, the threshold level is a mean and/or median expression level (median or absolute) of CDCA3 in a reference population, that, for example, have the same cancer type, subgroup, stage and/or grade as said mammal for which the expression level is determined. Additionally, the concept of a threshold level of expression should not be limited to a single value or result. In this regard, a threshold level of expression may encompass multiple threshold expression levels that could signify, for example, a high, medium, or low probability of, for example, response to the Bora-AurA-PLK1 pathway inhibitor or the inhibitor of the tyrosine kinase and optionally the further the anti-cancer agent, as described herein.

Suitably, an increased level of the CDCA3 protein or encoding nucleic acid indicates or correlates with increased responsiveness of the cancer to the Bora-AurA-PLK1 pathway inhibitor alone and/or in combination with the further anti-cancer agent; and/or a decreased level of CDCA3 protein or encoding nucleic acid indicates or correlates with decreased responsiveness of the cancer to the Bora-AurA-PLK1 pathway inhibitor alone and/or in combination with the further anti-cancer agent.

Accordingly, in particular examples, the subject's cancer demonstrates a reduced responsiveness or resistance to treatment with the Bora-AurA-PLK1 pathway inhibitor and optionally the further anti-cancer agent, such as in those cancers demonstrating a decreased expression level of a CDCA3 protein or encoding nucleic acid. In related examples, the subject's cancer demonstrates an increased responsiveness or sensitivity to treatment with the Bora-AurA-PLK1 pathway inhibitor and optionally the further anti-cancer agent, such as in those cancers demonstrating a decreased expression level of a CDCA3 protein or encoding nucleic acid. In such examples, and without being bound by any theory, it is believed that relatively high CDCA3 expression levels promote G2 checkpoint maintenance and mitotic entry by way of increased PLK1 activity or signaling in these cancers. Hence, targeted inhibition of this compensatory mechanism by an inhibitor of the Bora-AurA-PLK1 pathway or axis (e.g., a Bora inhibitor, an Aurora Kinase A inhibitor and/or a PLK1 inhibitor, as are known in the art) can assist in overcoming, at least in part, resistance to such further anti-cancer agents, including chemotherapeutic agents, such as a platinum-based chemotherapeutic agent (e.g., cisplatin).

In view of the above, the present method may further include the step of treating the cancer in the subject. By way of example, this can include administering to the subject a therapeutically effective amount of the Bora-AurA-PLK1 pathway inhibitor alone and/or in combination with the further anti-cancer agent, such as a chemotherapeutic agent, when the expression level of the CDCA3 protein or encoding nucleic acid, such as an increased expression level thereof, indicates or correlates with relatively increased responsiveness of the cancer to Bora-AurA-PLK1 pathway inhibitor alone and/or in combination with the further the anti-cancer agent. Thus, in various examples, the present method includes administering to the subject a therapeutically effective amount of the Bora-AurA-PLK1 pathway inhibitor alone and/or in combination with the further anti-cancer agent when an increased expression level of the CDCA3 protein or encoding nucleic acid is determined.

As used herein, the term “therapeutically effective amount” describes a quantity of a specified agent (e.g., an anti-cancer agent), such as a Bora-AurA-PLK1 pathway inhibitor, an inhibitor of a tyrosine kinase and/or a further anti-cancer agent, sufficient to achieve a desired effect in a subject being treated with that agent. For example, this can be the amount of a composition comprising one or more agents that are necessary to reduce, alleviate and/or prevent a cancer or cancer associated disease, disorder or condition. In some examples, a “therapeutically effective amount” is sufficient to reduce or eliminate a symptom of a cancer. In other examples, a “therapeutically effective amount” is an amount sufficient to achieve a desired biological effect, for example an amount that is effective to decrease or prevent cancer growth and/or metastasis or overcome resistance to and/or enhance the anti-cancer activity of the Bora-AurA-PLK1 pathway inhibitor and optionally the further anti-cancer agent.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

The terms “determining”, “measuring”, “evaluating”, “assessing” and “assaying” are used interchangeably herein and may include any form of measurement known in the art, such as those described hereinafter.

Determining, assessing, evaluating, assaying or measuring nucleic acids of CDCA3, such as RNA, mRNA and cDNA, may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-13 replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a cell or tissue source. In other examples, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

Determining, assessing, evaluating, assaying or measuring protein levels of CDCA3 may be performed by any technique known in the art that is capable of detecting cell- or tissue-expressed proteins whether on the cell surface or intracellularly expressed, or proteins that are isolated, extracted or otherwise obtained from the cell or tissue source. These techniques include antibody-based detection that uses one or more antibodies which bind the protein, electrophoresis, surface plasmon resonance (SPR), isoelectric focussing, protein sequencing, chromatographic techniques and mass spectroscopy and combinations of these, although without limitation thereto. Antibody-based detection may include flow cytometry using fluorescently-labelled antibodies that bind CDCA3, ELISA, immunoblotting, immunoprecipitation, in situ hybridization, immunohistochemistry and immunocytochemistry, although without limitation thereto. Suitable techniques may be adapted for high throughput and/or rapid analysis such as using protein arrays such as a TissueMicroArray™ (TMA), MSD MultiArrays™ and multiwell ELISA, although without limitation thereto.

It will be appreciated that determining the expression of CDCA3 may include determining both the nucleic acid levels thereof, such as by nucleic acid amplification and/or nucleic acid hybridization, and the protein levels thereof.

In certain examples, a gene expression level of CDCA3 may be assessed indirectly by the measurement of a non-coding RNA, such as miRNA, that regulate gene expression. MicroRNAs (miRNAs or miRs) are post-transcriptional regulators that bind to complementary sequences in the 3′ untranslated regions (3′ UTRs) of target mRNA transcripts, usually resulting in gene silencing. miRNAs are short RNA molecules, on average only 22 nucleotides long. The human genome may encode over 1000 miRNAs, which may target about 60% of mammalian genes and are abundant in many human cell types. Each miRNA may alter the expression of hundreds of individual mRNAs. In particular, miRNAs may have multiple roles in negative regulation (e.g., transcript degradation and sequestering, translational suppression) and/or positive regulation (e.g., transcriptional and translational activation). Additionally, aberrant miRNA expression has been implicated in various types of cancer.

Further aspects of the present disclosure relate to treatment of cancer in a subject.

In one particular aspect, the cancer treatment described herein is performed in conjunction with determining an expression level of CDCA3 protein or encoding nucleic acid in one or a plurality of cancer cells, tissues or organs of the subject, and based on the determination made, initiating, continuing, modifying or discontinuing the cancer treatment, wherein the cancer treatment comprises administration of a Bora-AurA-PLK1 pathway inhibitor.

Suitably, the cancer treatment comprises administration of a Bora-AurA-PLK1 pathway inhibitor and a further anti-cancer agent. Accordingly, in one form the present disclosure provides a method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of a Bora-AurA-PLK1 pathway inhibitor and optionally a further anti-cancer agent.

In this regard, it would be appreciated that those methods described herein for predicting the responsiveness of a cancer to a Bora-AurA-PLK1 pathway inhibitor and optionally a further anti-cancer agent, such as those hereinbefore described, may further include the step of administering to the mammal a therapeutically effective amount of the Bora-AurA-PLK1 pathway inhibitor and optionally the further anti-cancer agent.

In a related aspect, the present disclosure relates to a method of treating cancer in a subject, said method including the step of administering a therapeutically effective amount of a Bora-AurA-PLK1 pathway inhibitor to the subject in which a level of CDCA3 protein or encoding nucleic acid has been determined in one or a plurality of cancer cells, tissues or organs of the subject that indicates or correlates with increased responsiveness of the cancer to the Bora-AurA-PLK1 pathway inhibitor.

In particular examples, an increased level of CDCA3 protein or encoding nucleic acid has been determined in the one or plurality of cancer cells, tissues or organs of the subject.

Suitably, the method further includes administering a therapeutically effective amount of a further anti-cancer agent to the subject. In this regard, the level of CDCA3 protein or encoding nucleic acid can indicate or correlate with increased responsiveness of the cancer to the Bora-AurA-PLK1 pathway inhibitor and the further anti-cancer agent.

In particular examples, the Bora-AurA-PLK1 pathway inhibitor is or comprises a PLK1 inhibitor, such as those hereinbefore described. In some examples, the anti-cancer agent is or comprises a chemotherapeutic agent, such as those provided herein.

In various examples, the Bora-AurA-PLK1 pathway inhibitor and optionally the further anti-cancer agent or cancer treatment is administered when the CDCA3 expression level indicates or correlates with increased responsiveness of the cancer thereto. In this regard, the present method may further include the step of administering to the subject a therapeutically effective amount of the Bora-AurA-PLK1 pathway inhibitor and optionally the further anti-cancer agent, such as a chemotherapeutic agent, when an increased level of CDCA3 protein or encoding nucleic acid is determined.

In particular examples, the Bora-AurA-PLK1 pathway inhibitor is administered (i) prior to; (ii) after; or (iii) simultaneously with, the administration of the further anti-cancer agent (e.g., the chemotherapeutic agent). In various examples, administration of the agent that inhibits or prevents the expression and/or activity of Bora-AurA-PLK1 pathway, and the administration of the anti-cancer agent (either sequentially or concurrently) results in treatment or prevention of cancer that is greater than such treatment or prevention from administration of either the said agent or the anti-cancer agent in the absence of the other.

In another related aspect, the present disclosure provides a method of treating cancer in a subject, said method including the step of administering a therapeutically effective amount of an agent that increases the expression and/or activity of CDCA3 and a Bora-AurA-PLK1 pathway inhibitor to the subject.

Suitably, a decreased level of CDCA3 protein or encoding nucleic acid has been determined in one or a plurality of cancer cells, tissues or organs of the subject. As such, the present method may include the earlier step of determining an expression level of a CDCA3 protein or encoding nucleic acid in the one or plurality of cancer cells, tissues or organs of the subject.

Suitably, the method further includes administering a therapeutically effective amount of a further anti-cancer agent, such as those hereinbefore described, to the subject. In particular examples, the further anti-cancer agent is a chemotherapeutic agent, such as a platinum-based chemotherapeutic agent. In some examples, the further anti-cancer agent is an inhibitor of a tyrosine kinase, such as an EGFR inhibitor.

The agent that increases the expression and/or activity of CDCA3 can be any as are known in the art. By way of example, the agent may be an inhibitor of CDCA3 degradation and/or phosphorylation. To this end, the agent can be an inhibitor of APC/C, Cdh1, casein kinase 2 (CK2) or any combination thereof. In particular examples, the agent the increases the expression and/or activity of CDCA3 is a CK2 inhibitor. Exemplary CK2 inhibitors include CX-4945, CX5011, BMS-595, BMS-211, POM, Tetrabromobenzotriazole (TBB), DMAT, TMCB, TTP 22, (E) -3-(2,3,4,5-tetrabromophenyl) acrylic acid (TBCA) and ellagic acid.

Suitably, the various agents, anti-cancer agents or cancer treatments described herein are administered to a subject as a pharmaceutical composition comprising a pharmaceutically-acceptable carrier, diluent or excipient. In this regard, any dosage form and route of administration, such as those provided therein, may be employed for providing a subject with the composition of the present disclosure.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the present disclosure. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present disclosure suitable for oral or parenteral administration may be presented as discrete units such as capsules, sachets or tablets each containing a pre-determined amount of one or more therapeutic agents of the present disclosure, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the present disclosure with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present disclosure, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

It will be appreciated that the treatment regimes described herein may further include the administration of additional cancer treatments, as are known in the art. Such additional cancer treatments may include drug therapy, chemotherapy, antibody, nucleic acid and other biomolecular therapies, radiation therapy, surgery, nutritional therapy, relaxation or meditational therapy and other natural or holistic therapies, although without limitation thereto. Generally, drugs, biomolecules (e.g., antibodies, inhibitory nucleic acids such as siRNA) or chemotherapeutic agents are referred to herein as “anti-cancer therapeutic agents” or “anti-cancer agents”.

It is envisaged that the various agents, anti-cancer agents or cancer treatments described herein can be formulated as discrete doses, such as in the form of a kit. Such a kit may further comprise a package insert comprising printed instructions for simultaneous, concurrent, sequential, successive, alternate or separate use of the agents in the treatment, amelioration and/or prevention of cancer, as described herein, in a patient in need thereof. Accordingly, the aforementioned kits are suitably for use in a method of treating, ameliorating and/or preventing cancer, inclusive of one or more symptoms, consequences, sequelae or complications thereof, as described herein.

Instructions supplied in the kits of the present disclosure are typically written instructions on a label or package insert (e.g., a paper sheet included in the kit), but machine-readable instructions (e.g., instructions carried on a magnetic or optical storage disk) are also acceptable. The instructions relating to the use of the agents described herein, generally include information as to dosage, dosing schedule, and route of administration for the intended treatment. The kit may further comprise a description of selecting an individual suitable for treatment.

Alternatively, the various therapeutic agents described herein can be formulated together in a composition that optionally includes a pharmaceutically acceptable carrier, excipient or diluent.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

In particular examples, the methods described herein provide a “companion diagnostic” with respect to the cancer treatment, whereby the expression level of CDCA3 provides information to a clinician or the like that is used for the safe and/or effective administration of said cancer treatment.

Suitably, the cancer is of a type hereinbefore described, albeit without limitation thereto. In some examples, the cancer is or comprises lung cancer. In other examples, the cancer is or comprises breast cancer.

In yet another aspect, the present disclosure provides a kit for predicting the responsiveness of a cancer to treatment with a Bora-AurA-PLK1 pathway inhibitor in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the Bora-AurA-PLK1 pathway inhibitor.

Suitably, the expression level of the CDCA3 protein or encoding nucleic acid indicates or correlates with increased or decreased responsiveness of the cancer to the Bora-AurA-PLK1 pathway inhibitor and a further anti-cancer agent.

Accordingly, in one form, the present disclosure provides a kit for predicting the responsiveness of a cancer to treatment with a Bora-AurA-PLK1 pathway inhibitor and optionally a further anti-cancer agent in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the Bora-AurA-PLK1 pathway inhibitor and optionally the further anti-cancer agent.

Suitably, an increased level of CDCA3 protein or encoding nucleic acid indicates or correlates with increased responsiveness of the cancer to the Bora-AurA-PLK1 pathway inhibitor alone and/or in combination with the further anti-cancer agent; and/or a decreased level of CDCA3 protein or encoding nucleic acid indicates or correlates with decreased responsiveness of the cancer to the Bora-AurA-PLK1 pathway inhibitor alone and/or in combination with the further anti-cancer agent.

Suitably, the Bora-AurA-PLK1 pathway inhibitor is or comprises a PLK1 inhibitor.

Suitably, the further anti-cancer agent is or comprises a chemotherapeutic agent. In various examples, the chemotherapeutic agent is or comprises a platinum-based chemotherapeutic agent, such as those described herein. In some examples, the chemotherapeutic agent is or comprises cisplatin and/or a derivative thereof.

In some examples, the kit further comprises reference data for correlating the expression level of the CDCA3 protein or encoding nucleic acid and responsiveness of the cancer to the anti-cancer agent.

In particular examples, the reference data is on a computer-readable medium (e.g., software embodying or utilised by any one or more of the methodologies or functions described herein). The computer-readable medium can be included on a storage device, such as a computer memory (e.g., hard disk drives or solid state drives) and may comprise computer readable code components that when selectively executed by a processor implements one or more aspects of the present disclosure.

Suitably, the present kit is for use in the method of the aforementioned aspects.

In particular examples, the present kit provides a “companion diagnostic” whereby information with respect to CDCA3 expression levels are utilized by a clinician or similar for the safe and effective administration of a Bora-AurA-PLK1 pathway inhibitor with or without a further anti-cancer agent.

It will be appreciated from the foregoing that the present disclosure provides methods that predict the responsiveness of a cancer to a Bora-AurA-PLK1 pathway inhibitor alone and/or in combination with a further anti-cancer treatment, and more particularly a chemotherapeutic agent. Particular broad examples of the present disclosure include the step of treating the patient following predicting the responsiveness of the cancer to the Bora-AurA-PLK1 pathway inhibitor and optionally the further anti-cancer treatment. Accordingly, these examples relate to using information obtained about the predicted responsiveness of the cancer to anti-cancer treatment to thereby construct and implement an anti-cancer treatment regime for the patient. In various examples, this is personalized to a particular patient so that the treatment regime is optimized for that particular patient.

In yet another aspect, the present disclosure provides a method of predicting the responsiveness of a cancer to treatment with an inhibitor of a tyrosine kinase in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the inhibitor of the tyrosine kinase.

Suitably, the present method is for predicting the responsiveness of the cancer to treatment with the inhibitor of the tyrosine kinase and a further anti-cancer agent. As such, in one particular form, the present disclosure relates to a method of predicting the responsiveness of a cancer to treatment with an inhibitor of a tyrosine kinase and optionally a further anti-cancer agent in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the inhibitor of the tyrosine kinase and optionally the further anti-cancer agent in the subject.

In particular examples, an increased level of the CDCA3 protein or encoding nucleic acid indicates or correlates with increased responsiveness of the cancer to the inhibitor of the tyrosine kinase alone and/or in combination with the further anti-cancer agent; and/or a decreased level of CDCA3 protein or encoding nucleic acid indicates or correlates with decreased responsiveness of the cancer to the inhibitor of the tyrosine kinase alone and/or in combination with the further anti-cancer agent.

Suitably, the method of the present aspect includes the further step of treating the cancer in the subject. In this regard, the cancer treatment can include administration of the inhibitor of the tyrosine kinase. In particular examples, the cancer treatment includes administration of the inhibitor of the tyrosine kinase in combination with the further anti-cancer agent. In one example, an increased level of CDCA3 protein or encoding nucleic acid indicates or correlates with increased responsiveness of the cancer to the inhibitor of the tyrosine kinase alone and/or in combination with the further anti-cancer agent, the method further includes the step of administering to the subject a therapeutically effective amount of the inhibitor of the tyrosine kinase and optionally the further anti-cancer agent.

In a further related aspect, the present disclosure provides a method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of an inhibitor of a tyrosine kinase.

Suitably, the cancer treatment comprises administration of the inhibitor of the tyrosine kinase and a further anti-cancer agent. Accordingly, in one form, the present disclosure describes a method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of an inhibitor of a tyrosine kinase and optionally a further anti-cancer agent.

In a related aspect, the present disclosure relates to a method of treating cancer in a subject, said method including the step of administering a therapeutically effective amount of an inhibitor of a tyrosine kinase to the subject in which a level of CDCA3 protein or encoding nucleic acid has been determined in one or a plurality of cancer cells, tissues or organs of the subject that indicates or correlates with increased responsiveness of the cancer to the inhibitor of the tyrosine kinase.

Suitably, the method further includes administering a therapeutically effective amount of a further anti-cancer agent to the subject. In this regard, the level of CDCA3 protein or encoding nucleic acid can indicate or correlate with increased responsiveness of the cancer to the inhibitor of the tyrosine kinase and the further anti-cancer agent.

In various examples, the inhibitor of the tyrosine kinase and optionally the further anti-cancer agent or cancer treatment is administered when the CDCA3 expression level indicates or correlates with increased responsiveness of the cancer thereto. In this regard, the present method may further include the step of administering to the subject a therapeutically effective amount of the inhibitor of the tyrosine kinase alone or in combination with the further anti-cancer agent, such as a chemotherapeutic agent and/or a Bora-AurA-PLK1 pathway inhibitor, when an increased level of CDCA3 protein or encoding nucleic acid is determined. Additionally, the present method may further include the step of not administering to the subject a therapeutically effective amount of the inhibitor of the tyrosine kinase and optionally the further anti-cancer agent, such as a chemotherapeutic agent and/or a Bora-AurA-PLK1 pathway inhibitor, when a decreased level of CDCA3 protein or encoding nucleic acid is determined.

Suitably, for the three aforementioned aspects, a decrease in the expression level of the CDCA3 protein or encoding nucleic acid in the one or plurality of cancer cells, tissues or organs of the subject with administration of the inhibitor of the tyrosine kinase and optionally the further anti-cancer agent indicates or correlates with increased responsiveness of the cancer thereto; and/or an increase or no change in the expression level of the CDCA3 protein or encoding nucleic acid in the one or plurality of cancer cells, tissues or organs of the subject with administration of the inhibitor of the tyrosine kinase and optionally the further anti-cancer agent indicates or correlates with decreased responsiveness or resistance of the cancer thereto.

In particular examples, the inhibitor of the tyrosine kinase is administered (i) prior to; (ii) after; or (iii) simultaneously with, the administration of the further anti-cancer agent (e.g., the chemotherapeutic agent and/or the Bora-AurA-PLK1 pathway inhibitor). In various examples, administration of the inhibitor of the tyrosine kinase, and the administration of the further anti-cancer agent (either sequentially or concurrently) results in treatment or prevention of cancer that is greater than such treatment or prevention from administration of either the said inhibitor or the anti-cancer agent in the absence of the other.

Suitably, for the three aforementioned aspects, the inhibitor of the tyrosine kinase is or comprises an EGFR inhibitor, such as that hereinbefore described. In some examples, the EGFR inhibitor is erlotinib. In particular examples, the EGFR inhibitor is osimertinib.

In various examples of the three prior aspects, the further anti-cancer agent suitably is or comprises a chemotherapeutic agent, such as a platinum-based chemotherapeutic agent and including those provided herein.

In other examples of the three above aspects, the further anti-cancer agent can be or comprise a Bora-AurA-PLK1 pathway inhibitor, and more particularly a PLK1 inhibitor, such as those described herein.

As such, in various examples, the treatment described herein includes an EGFR inhibitor and a Bora-AurA-PLK1 pathway inhibitor. In some examples, the treatment described herein includes an EGFR inhibitor and a PLK1 inhibitor. In certain examples, the treatment described herein includes an EGFR inhibitor and a chemotherapeutic agent, such as a platinum-based chemotherapeutic agent. In particular examples, the treatment described herein includes an EGFR inhibitor, a PLK1 inhibitor and a chemotherapeutic agent.

In another aspect, the present disclosure provides a method of treating cancer in a subject, said method including the step of administering a therapeutically effective amount of an agent that increases the expression and/or activity of CDCA3 and an inhibitor of a tyrosine kinase to the subject.

Suitably, a decreased level of CDCA3 protein or encoding nucleic acid has been determined in one or a plurality of cancer cells, tissues or organs of the subject. As such, the present method may include the earlier step of determining an expression level of a CDCA3 protein or encoding nucleic acid in the one or plurality of cancer cells, tissues or organs of the subject.

The agent that increases the expression and/or activity of CDCA3 and the inhibitor of the tyrosine kinase may be that as hereinbefore described. In particular examples, the agent that increases the expression and/or activity of CDCA3 is a CK2 inhibitor, such as that described herein. In various examples, the inhibitor of the tyrosine kinase is an EGFR inhibitor.

Suitably, the method further includes administering a therapeutically effective amount of a further anti-cancer agent, such as those hereinbefore described, to the subject.

In still yet another aspect, the present disclosure provides a kit for predicting the responsiveness of a cancer to treatment with an inhibitor of a tyrosine kinase in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the inhibitor of the tyrosine kinase.

Suitably, the expression level of the CDCA3 protein or encoding nucleic acid indicates or correlates with increased or decreased responsiveness of the cancer to the inhibitor of the tyrosine kinase and a further anti-cancer agent.

Accordingly, in one form the present disclosure relates to a kit for predicting the responsiveness of a cancer to treatment with an inhibitor of a tyrosine kinase and optionally a further anti-cancer agent in a subject, the kit comprising at least one reagent capable of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to the inhibitor of the tyrosine kinase and optionally the further anti-cancer agent.

In particular examples, an increased level of CDCA3 protein or encoding nucleic acid indicates or correlates with increased responsiveness of the cancer to the inhibitor of the tyrosine kinase and optionally the further anti-cancer agent; and/or a decreased level of CDCA3 protein or encoding nucleic acid indicates or correlates with decreased responsiveness of the cancer to the inhibitor of the tyrosine kinase and optionally the further anti-cancer agent.

Suitably, the present kit further includes a collection of data comprising correlation data or reference data for correlating the expression level of the CDCA3 protein or encoding nucleic acid and responsiveness of the cancer to the inhibitor of the tyrosine kinase and optionally the further anti-cancer agent, such as that hereinbefore described. In particular examples, the collection of data or reference data is on a computer-readable medium.

Suitably, the kit is for use in the method of the two aforementioned aspects.

In some examples of the three aforementioned aspects, the method or kit is or comprises a companion diagnostic.

Suitably, the inhibitor of the tyrosine kinase suitably is an EGFR inhibitor, such as that hereinbefore described.

In various examples, the further anti-cancer agent suitably is or comprises a chemotherapeutic agent, such as a platinum-based chemotherapeutic agent and including those provided herein.

In other examples, the further anti-cancer agent can be or comprise a Bora-AurA-PLK1 pathway inhibitor, and more particularly a PLK1 inhibitor, such as those described herein.

It is envisaged that one or more steps of the methods described herein may be automated or implemented by a computer.

By way of example, comparing an expression level of the CDCA3 protein and/or encoding nucleic acid with, for example, a reference or threshold level or value may be carried out manually or computer-assisted. Thus, the comparison may be carried out by a computer or computing device. The value of the determined or detected amount of CDCA3 in the sample from the subject and the reference amount can be, for example, compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired assessment in a suitable output format.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). In various examples, the subject is a human.

All computer programs, algorithms, protein and nucleic acid sequences (i.e., accession numbers), patent and scientific literature referred to herein is incorporated herein by reference.

The following non-limiting examples illustrate the methods and kit of the present disclosure. These examples should not be construed as limiting: the examples are included for the purposes of illustration only.

### EXAMPLE 1

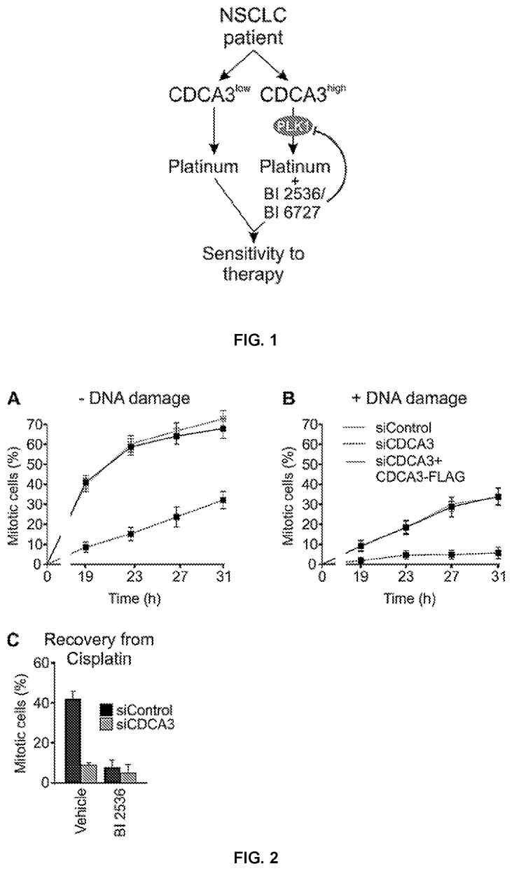

The present Example establishes cell division cycle associated protein 3 (CDCA3) expression as a NSCLC diagnostic to improve health outcomes for non-responders to platinum-based chemotherapy. The inventors have previously found that CDCA3 is a key cell cycle factor in NSCLC tumours [8]. The preliminary findings indicate that CDCA3low NSCLC is sensitive to platinum-agents while CDCA3high tumours do not respond to therapy. In these tumours, CDCA3 mediates activity of polo-like kinase-1 (PLK1). As such, CDCA3 expression could serve as a complementary diagnostic whereby CDCA3low patients receive front-line platinum therapy while CDCA3high patients receive platinum therapy in combination with a PLK1 inhibitor (FIG. 1).

The present Example describes a novel strategy to combine PLK1 inhibitors (BI 2536/BI 6727) with platinum agents to treat NSCLC with CDCA3high expression. These PLK1 inhibitors are well tolerated and have completed phase I/II and reached phase III trial [9-12]. However, they have not proven efficacious as a monotherapy [13]. It is proposed that these agents will prove clinically useful in NSCLC patients when combined with platinum therapy, particularly with the use of elevated CDCA3 levels as a complementary diagnostic. Without being bound by any theory, it is believed that high CDCA3 expression (CDCA3high) in NSCLC is associated with a poor tumour response to therapy. High CDCA3 promotes PLK1 activity and checkpoint maintenance, thus by blocking PLK1 activity in these tumours, with specific, clinically tested PLK1 inhibitors, this will enhance patient tumour responsiveness to therapy and improve patient outcomes.

Progression through the cell cycle relies upon coordination of a complex network of proteins. Following genomic insult, cellular checkpoints during each stage of the cell cycle are engaged to halt cell cycle progression and allow faithful DNA repair [14, 15]. The G2 checkpoint and normal cell cycle entry into mitosis is primarily mediated by the master regulator CDK1 bound to the regulator subunit cyclin B1. Activity of CDK1-cyclinB1 is controlled by the kinases WEE1 and PLK1. The tyrosine kinase WEE1 functions to inhibit mitotic entry by phosphorylating CDK1 [16]. On the other hand, PLK1, which itself is activated by the kinase Aurora A [17], is required for mitotic entry and recovery from the G2 checkpoint by ensuring the timely dephosphorylation of CDK1 by the cdc25c phosphatase [18]. Deregulation of these CDK1 feedback loops and loss of an adequate G2 checkpoint results in genomic instability and ultimately cancer development.

Whilst loss of genomic stability is a key hallmark of cancer [19], current chemotherapy serves to exacerbate genome instability and induce tumour cell death. For example, the gold standard treatment for lung cancer, in particular NSCLC, is cisplatin-based chemotherapy. Cisplatin functions by forming DNA crosslinks that hinder the DNA repair process and push cells towards apoptosis [20, 21]. However, resistance to cisplatin is a major therapeutic problem [22]. Further understanding of the resistance mechanisms is critical to the development of new therapeutic intervention strategies. Given the abundant loss of the G1/S checkpoint in tumours (such as in p53 deficient tumour [23, 24]), G2/M cell cycle checkpoint factors might serve as diagnostics for chemotherapy response.

### Introduction to CDCA3

To identify novel genes that preserve NSCLC cell survival in response to cisplatin the present inventors performed bioinformatics and quantitative proteomics. These approaches identified CDCA3 as a candidate (APP1091589). CDCA3 is reported to function as part of a cullin-RING ubiquitin ligase (E3) complex to degrade the endogenous cell cycle inhibitor WEE1, thereby modulating the cell cycle [25]. CDCA3 levels are also regulated through the cell cycle by transcription and protein degradation during the G1 phase of the cell cycle [25, 26]. A role for CDCA3 in tumour biology [27] is emerging with up-regulated expression noted in liver cancer [28], oral squamous cell carcinoma tissues [29] and prostate cancer cells [30]. More recently, the inventors have also demonstrated that CDCA3 is commonly upregulated in NSCLC and that patients with elevated levels of CDCA3 in this disease have a poorer outcome 5 than patients with lower levels of CDCA3 [8]. However, little else is known about the function of CDCA3 in human cancers or whether this protein may prove useful clinically.

### CDCA3high Patients Do Not Respond to Adjuvant Platinum-Based Chemotherapy

To investigate the diagnostic potential for CDCA3, the present inventors stratified the UT Lung SPORE cohort data based on CDCA3 expression (CDCA3low=below median, CDCA3high=above median). In this trial, patients undergoing curative resection were either observed only (OBS) or received adjunct chemotherapy (ACT) [31]. It was found that therapy was as effective as observation only in CDCA3low patients (FIG. 2A). Strikingly, CDCA3high patients had a significantly poorer prognosis when treated with adjuvant platinum-based chemotherapy versus observation patients (FIG. 2B). In FIG. 8, it was further demonstrated that low CDCA3 levels enhance cisplatin sensitivity in lung cancer cell lines in vitro.

These data suggest that patients with elevated CDCA3 levels respond poorly to platinum-based therapy, with treatment providing no benefit while significantly reducing their quality of life. Employing CDCA3 as a diagnostic might improve chemotherapy response rates and health outcomes.

### CDCA3 is Required to Recover From Cisplatin-Induced Cell Cycle Arrest

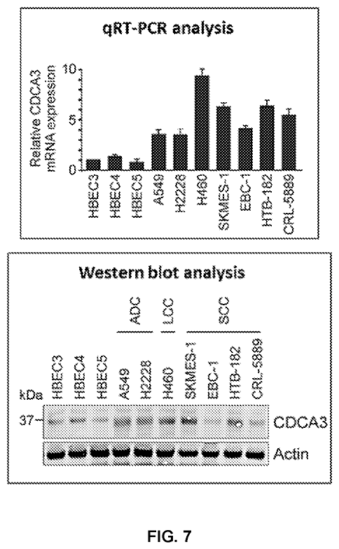

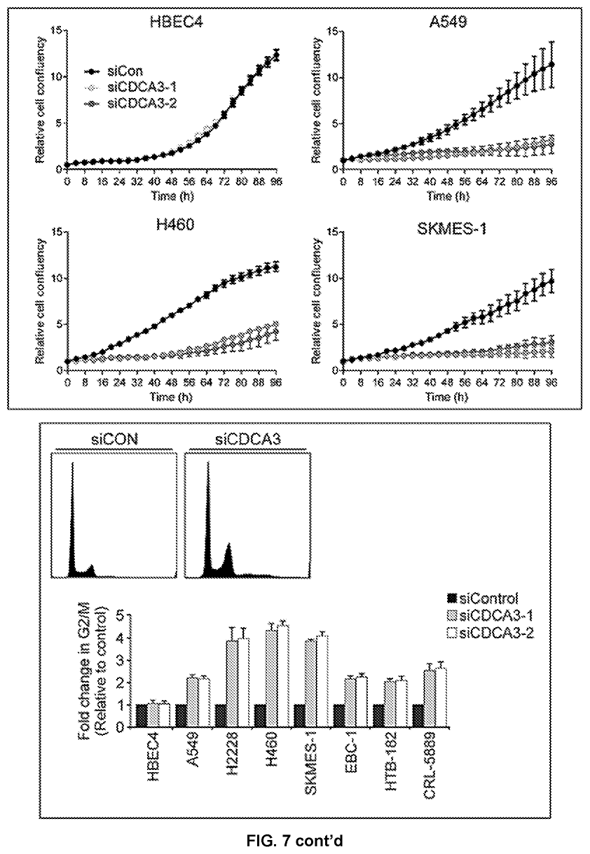

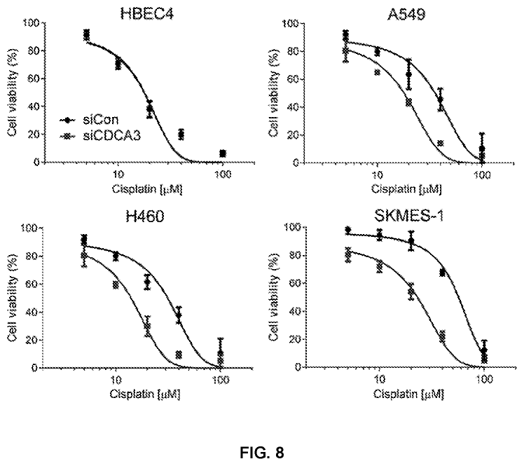

To investigate strategies to improve the health outcomes for patients with CDCA3high NSCLC tumours, that do not respond to therapy, the present inventors examined the cellular function of CDCA3. As this protein is reported to regulate cell proliferation (as confirmed by data in FIG. 7), the present inventors investigated its potential role in cell cycle progression from G2 into mitosis. To examine cell cycle progression, control cells, CDCA3-depleted cells or depleted cells ectopically expressing siRNA resistant CDCA3-FLAG were synchronized at the G1/S cell cycle boundary by a double thymidine block and released for seven hours (˜G2 phase). At this time cells were treated with or without cisplatin for 3.5 h and released into nocodazole containing media to capture cells in mitosis. Mitotic cells were determined by flow cytometry analysis of cells stained for Ser-10 phosphorylated histone H3. In undamaged cells, CDCA3 depletion slowed the progression of cells from G2 into mitosis (FIG. 2A). However, in cisplatin treated cells, CDCA3 depletion abolished mitotic progression with an inability to recover from G2 cell cycle arrest (FIG. 2B). These data indicate that CDCA3 is required for mitotic entry in unperturbed cells and also G2 checkpoint recovery in cells responding to DNA damage. As these observed cell cycle phenotypes are consistent with pharmacological inhibition or depletion of PLK1 [18], the present inventors sought to examine the possibility of a functional association between CDCA3 and this kinase during the G2 checkpoint. While CDCA3 depletion blocked mitotic entry and recovery from the cisplatin-induced G2 checkpoint (as per FIG. 2B), PLK1 inhibition with the small molecule BI 2536 blocked mitotic entry for both control and CDCA3-depleted cells (FIG. 2C). These data suggest that CDCA3 functions in the same pathway as PLK1 and is required for mitotic entry and recovery from the G2 checkpoint.

### CDCA3 Mediates Cell Cycle Progression by Interacting with PLK1 and Aurora A

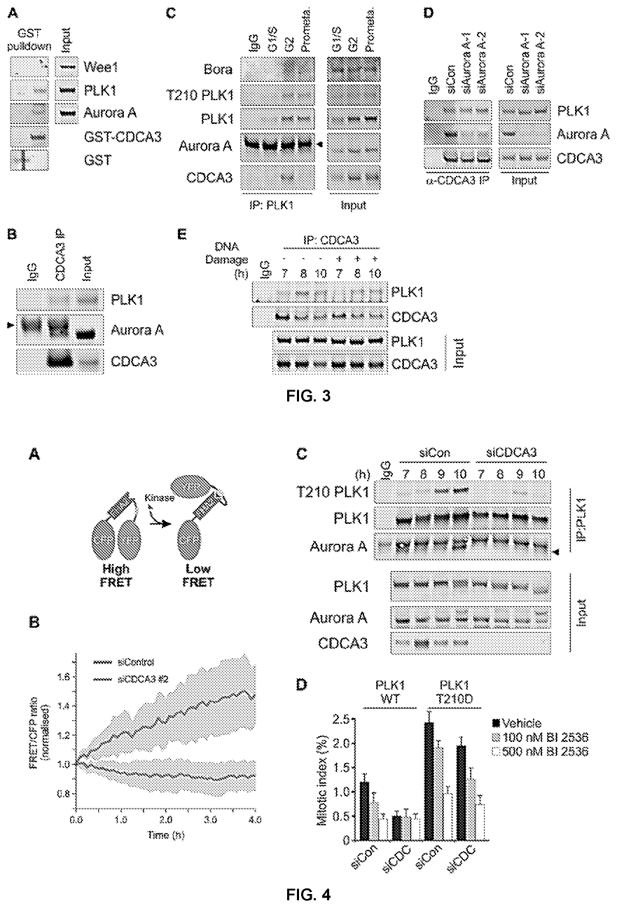

To determine the molecular regulation of CDCA3 and the G2 checkpoint, a GST-CDCA3 pulldown was performed to identify interaction partners. Whole cell lysates of A549 NSCLC cells synchronised in G2 and treated with or without cisplatin were incubated with GST-alone or GST-CDCA3 and subjected to Western blot analysis. Although CDCA3 is reported to modulate WEE1 through the SCF complex, the present inventors have been unable to experimentally verify that CDCA3 is capable of binding WEE1 (FIG. 3A) or components of the SCF complex (e.g. Cullin 1, data not shown). These findings are also consistent with a previous report that was unable to identify a functional link between CDCA3 and WEE1 [32]. The pulldown analysis indicated that CDCA3 is capable of associating with PLK1 and with Aurora A, the kinase required for timely PLK1 activation (FIG. 3A). Immunoprecipitation analysis of lysates from asynchronous cells confirmed that CDCA3 associates with PLK1 and Aurora A in vivo (FIG. 3B). Immunoprecipitation analysis of lysates from cells synchronised at the G1/S boundary, G2 and prometaphase further indicated that CDCA3 and PLK1 predominantly associate during the G2 phase of the cell cycle (FIG. 3C). To identify if CDCA3 associating with PLK1 was dependent upon Aurora A, which phosphorylates PLK1 at T210, cells were depleted of Aurora A using two different siRNAs and immunoprecipitation analysis was performed. As shown in FIG. 3D, depletion of Aurora A did not affect the association between CDCA3 and PLK1. Immunoprecipitation analysis of G2 synchronised A549 lysates also indicated that endogenous CDCA3 associates with PLK1 at early G2 (˜7-8 h post release from thymidine block) and late G2/early mitosis (˜10 h post release) in unperturbed cells (FIG. 3E, confirming FIG. 3C). Interestingly, activation of the G2 checkpoint by ionising radiation (IR)-induced DNA damage indicated that CDCA3 and PLK1 remain associated (FIG. 3E).

### CDCA3 is Required for Efficient Cellular PLK1 Activity

The present inventors next utilised an established FRET-based PLK1 biosensor to examine the level of PLK1 activity in cells depleted of CDCA3 in real-time by live cell spinning disc microscopy. FRET-based biosensors contain CFP and YFP fluorophores that are conjoint by a linker sequence which is phosphorylated by the kinase of interest [33]. In this instance, a FRET biosensor containing a c-jun sequence, which is a well characterised PLK1 substrate [18], was used. As shown in FIG. 4A, FRET occurs in a basal setting while phosphorylation of the linker sequence causes a conformational change separating the fluorophores and reducing FRET, thereby providing a readout for kinase activity. In live imaging of G2 synchronised scrambled siRNA control cells, an increase in CFP/YFP ratio is observed in late G2 at 3-4 h following commencement of imaging (equating to 9-10 h post release) indicative of increased PLK1 activity and consistent with the temporal activity profile for PLK1 (FIG. 4B; [18]). Interestingly, compared with control cells, depletion of CDCA3 resulted in a marked reduction in PLK1 activity (FIG. 4B). PLK1 is activated by phosphorylation of T210 by the kinase Aurora A. To determine if CDCA3 is required for PLK1 activity in G2 phase, the T210 phosphorylation status of PLK1 was examined in control versus CDCA3 depleted cells synchronised in G2 (7-10 h post release). As shown in FIG. 4C, depletion of CDCA3 markedly impairs the binding of endogenous PLK1 with Aurora A in G2. Consistent with the loss of Aurora A binding, markedly less T210 phospho-PLK1 was detected in CDCA3 depleted cells. The present inventors next introduced a constitutively active form of PLK1 (T210D mutant) to determine if CDCA3 depleted cells are capable of undergoing mitosis. Expression of active PLK1 (T210D) increased the mitotic index of both control and CDCA3 depleted cells (FIG. 4D). Mitotic entry was reduced by BI 2536 treatment in a dose dependent manner. These data highlight that CDCA3 is a necessary component for efficient PLK1 activity.

### Phosphorylation of CDCA3 by CDK1 Regulates Binding to, and Activity of PLK1

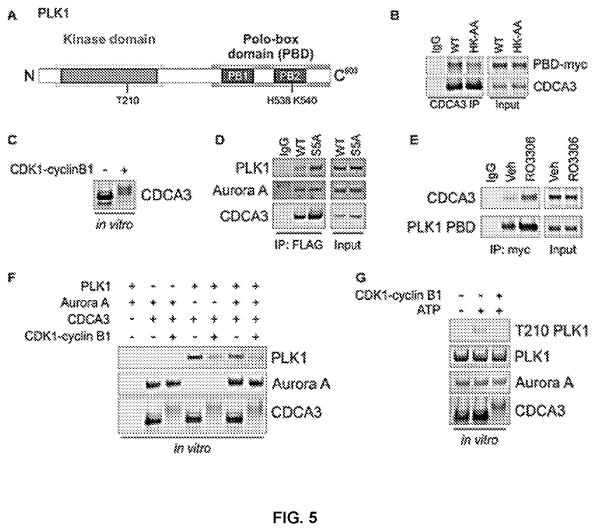

The present inventors next examined the mechanism regulating the association between CDCA3 and PLK1. The structure of PLK1 consists of an N-terminal kinase domain and a C-20 terminal polo-box domain (PBD; FIG. 5A). Classically, the PBD binds phosphorylated substrates requiring the residues His538 and Lys540. Using immunoprecipitation analysis of lysates from cells ectopically expressing CDCA3 and the PBD of PLK1, it was identified that CDCA3 associates with the PLK1 PBD independent of residues His538 and Lys540 (FIG. 5B). The present inventors next investigated the post-translational modification of CDCA3. Like most cell cycle regulated proteins, it was identified that CDCA3 is a cyclin-dependent kinase 1 (CDK1)-cyclin B1 substrate. Incubation of recombinant CDCA3 with recombinant CDK1-cyclin B1 caused a large molecular weight shift by SDS-PAGE analysis, indicative of phosphorylation (FIG. 5C). By performing mass spectrometry analysis, it was identified that CDK1-cyclin B1 phosphorylates CDCA3 at residues Thr10, Ser29, Ser68, Thr76 and Ser87 (data not shown). Mutation of these residues to alanines to block CDCA3 phosphorylation promoted the association between CDCA3 and PLK1 (FIG. 5D). Consistently, inhibition of CDK1 with the drug R03306 to block CDCA3 phosphorylation also induced an increase in association between CDCA3 and PLK1 (FIG. 5E). These data suggest that phosphorylation of CDCA3 negatively regulates its binding to PLK1. Indeed, in in vitro pull-down analysis, it was identified that CDCA3 pre-phosphorylated by CDK1-cyclin B1 was not able to bind PLK1 to the same level of unmodified CDCA3 (FIG. 5F). Phosphorylation did not affect CDCA3 binding to Aurora A. The present inventors next assessed in vitro PLK1 activation assays by incubating recombinant CDCA3, either unmodified or phosphorylated, with recombinant PLK1 in the presence of active Aurora A. As shown in FIG. 5G, while unmodified CDCA3 enabled Aurora A-mediated PLK1 activation, pre-phosphorylated CDCA3 prevented in vitro activation of PLK1.

### CDCA3 Levels Correlate with NSCLC Sensitivity to Platinum-Based Therapy

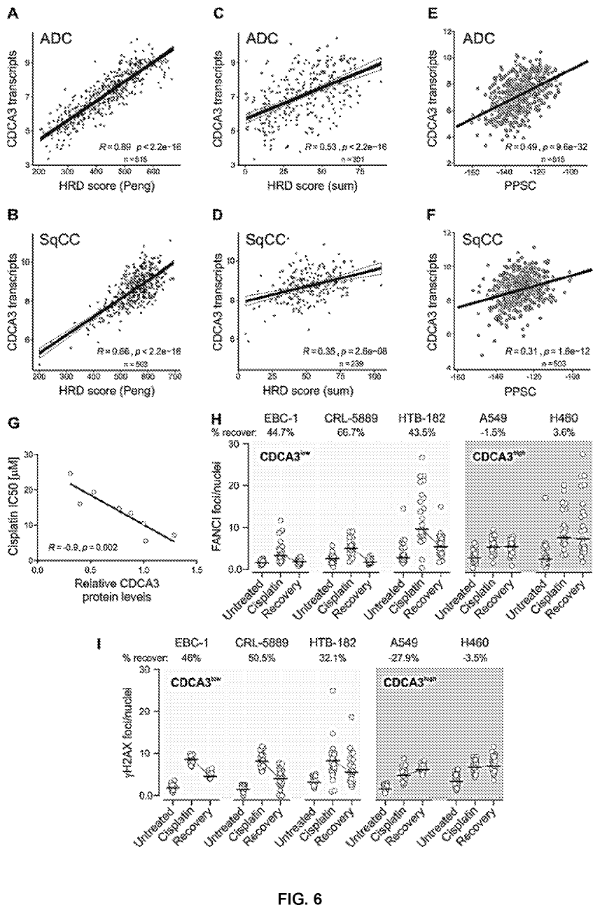

Cisplatin-based regimens are the most commonly employed treatments of NSCLC [6]. Novel therapeutic strategies and complementary diagnostics are needed to prevent cisplatin resistance and identify those NSCLC patients who will respond best to therapy. To assess the utility of CDCA3 as a tool to identify platinum-based chemotherapy response in NSCLC, the present inventors evaluated available patient data for correlations with gene signatures or DNA-based measures of genome instability. Defective DNA repair is associated with improved response to DNA-damaging therapeutics such as platinum agents in lung cancer¹ and other solid malignancies². To this end, the present inventors undertook bioinformatics analyses of TCGA datasets to correlate CDCA3 transcript levels with a homologous recombination deficiency (HRD) score in the ADC and SqCC NSCLC histologies. As shown in FIG. 6, CDCA3 expression significantly correlated with a HRD score in ADC and SqCC disease determined firstly by a multigene signature representative of HR deficiencies³ (FIG. 6a,b) and the unweighted sum of three genomic scores, namely loss of heterozygosity, telomeric allelic imbalance and large scale state transitions⁴ (FIG. 6c,d). In each analysis, associations between CDCA3 expression and HRD score were stronger in ADC disease.

The present inventors next evaluated correlations between CDCA3 and another multigene signature predictive of chemotherapeutic drug response, termed the pharmacogenomic predictor of sensitivity to chemotherapy (PPSC)⁵. While first applied in breast cancers to predict pathologic complete response (pCR) to chemotherapy⁵, this analysis has been used to assess NSCLC cases⁶ and shows utility for DNA-damaging therapeutics. In ADC (FIG. 6e) and SqCC (FIG. 6f) disease, CDCA3 expression positively correlated with the PPSC signature with respective Spearman correlation coefficients of r=0.49 and r=0.31.

Given the significant associations in the clinical data, the correlation between CDCA3 protein levels was evaluated, as determined by western blot analysis⁷, with cisplatin potency (IC₅₀ values) in a panel of eight NSCLC cell lines. Cisplatin induced a dose-dependent reduction in cell viability across all cell lines tested (data not shown). Consistent with the bioinformatics analyses, CDCA3 protein levels strongly correlated with the sensitivity of NSCLC cell lines to cisplatin (r=−0.9; FIG. 6g), whereby CDCA3^{high} cell lines exhibited greatest cisplatin sensitivity. Of note, the CDCA3^{high} cell lines A549 and H460, versus three CDCA3^{low} cell lines, exhibited persistent nuclear foci of the DNA damage markers FANCI (FIGS. 6h) and γH2AX (FIG. 61) following an 8 hour recovery from cisplatin exposure. Persistent DNA damage foci at the times tested, determined by immunofluorescence image analysis of high-throughput microscopy, point to a reduced DNA damage repair capacity. Collectively, these results suggest that CDCA3 expression correlates with DNA-based and functional measures of genome instability where CDCA3^{high} tumours are more sensitive to platinum agents.

### PLK1 Inhibitors Are More Potent in CDCA3^{high} Tumours and Are Additive with Platinum-Based Therapy

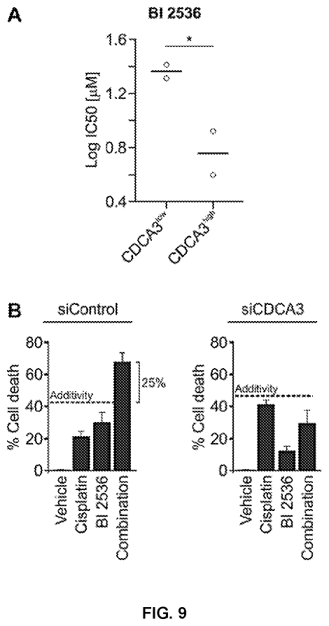

The current findings indicate that CDCA3^{low} patient tumours do not respond to platinum therapy and the cellular function for CDCA3 is to ensure timely PLK1 activity. The present data also indicate that CDCA3 is required for the efficient activation of PLK1 in cells. The present inventors next sought to examine whether PLK1 inhibition in CDCA3^{high} NSCLC cells is a viable strategy to further enhance cisplatin responsiveness, potentially as a strategy to prevent or delay drug resistance. This was first tested in NSCLC cells stratified by high versus low levels of CDCA3 protein. Potency values (IC₅₀) were generated by assessing cell viability for escalating doses of BI 2536. As shown in FIG. 9A, BI 2536 was significantly more potent in cells expressing higher endogenous CDCA3 levels versus CDCA3 low cells.

The present inventors next tested whether PLK1 blockade enhanced cisplatin sensitivity in NSCLC cells expressing CDCA3. As shown in FIG. 9B, BI 2536 induced ˜30% cell death as a monotherapy in control cells but was significantly less effective in CDCA3 25 depleted cells (˜13%), pointing to the possible functional association between CDCA3 and PLK1. Interestingly, combination of cisplatin and BI 2536 resulted in ˜70% cell death and was significantly more effective than cisplatin alone in CDCA3 expressing NSCLC cells. Again, BI 2536 was less effective in combination with cisplatin in CDCA3low cells and did not reach the theoretical additivity line. These data point to the possibility that PLK1 inhibitors are most effective in CDCA3high expressing tumours versus CDCA3low tumours.

### Discussion

The present findings support an approach to combine cisplatin with PLK1 inhibitors (e.g., BI 2536 or BI 6727) to enhance response to platinum-based therapy in those NSCLC patients overexpressing CDCA3. PLK1 is often overexpressed across human cancers correlating with poor patient prognosis and aggressive tumours [34]. The Boehringer Ingelheim PLK1 inhibitors BI 2536 (1st generation inhibitor) and BI 6727 (Volasertib) have acceptable safety profiles while BI 6727 appears to be the most effective available PLK1 inhibitor having entered phase III clinical trial [9]. However, these drugs have limited response rates as a monotherapy (<20% response rate; [12]), with combination therapy and complementary diagnostics suggested to improve efficacy.

### REFERENCES

1 Kadouri, L. et al. Homologous recombination in lung cancer, germline and somatic mutations, clinical and phenotype characterization. Lung Cancer 137, 48-51, doi:10.1016/j.lungcan.2019.09.008 (2019).
2 Telli, M. L. et al. Homologous Recombination Deficiency (HRD) Score Predicts Response to Platinum-Containing Neoadjuvant Chemotherapy in Patients with Triple-Negative Breast Cancer. Clin Cancer Res 22, 3764-3773, doi:10.1158/1078-0432.CCR-15-2477 (2016).
3 Peng, G. et al. Genome-wide transcriptome profiling of homologous recombination DNA repair. Nat Commun 5, 3361, doi:10.1038/ncomms4361 (2014).
4 Marquard, A. M. et al. Pan-cancer analysis of genomic scar signatures associated with homologous recombination deficiency suggests novel indications for existing cancer drugs. Biomark Res 3, 9, doi:10.1186/s40364-015-0033-4 (2015).
5 Hess, K. R. et al. Pharmacogenomic predictor of sensitivity to preoperative chemotherapy with paclitaxel and fluorouracil, doxorubicin, and cyclophosphamide in breast cancer. J Clin Oncol 24, 4236-4244, doi:10.1200/JCO.2006.05.6861 (2006).
6 Shukla, A. et al. Chromosome arm aneuploidies shape tumour evolution and drug response. Nat Commun 11, 449, doi:10.1038/s41467-020-14286-0 (2020).
7 Adams, M. N. et al. Expression of CDCA3 Is a Prognostic Biomarker and Potential Therapeutic Target in Non-Small Cell Lung Cancer. J Thorac Oncol 12, 1071-1084, doi:10.1016/j.jtho.2017.04.018 (2017).

### EXAMPLE 2

The present Example further establishes cell division cycle associated protein 3 (CDCA3) expression as a prognostic tool in breast cancer patients to improve health outcomes for non-responders to platinum-based chemotherapy.

### CDCA3 Expression is Prognostic in Breast Cancer with Elevated Expression in Triple-Negative Breast Cancer

The current data indicate that CDCA3 expression is prognostic in NSCLC. The present inventors have also identified that CDCA3 transcript levels are also prognostic in other solid cancers. For example, univariate Kaplan-Meier analysis across 1402 cases of breast cancer cases indicated that, consistent with NSCLC, patients with elevated CDCA3 had a poorer outcome than those patients with lower levels of CDCA3 (HR=1.77, CI=1.43-2.19, log rank p=<1⁻⁴; FIG. 9A).

The present inventors next assessed CDCA3 protein expression across the subtypes of breast cancer using an extensive panel of 23 in vitro breast cancer cell lines. As shown in FIG. 9B, CDCA3 protein was markedly elevated in cell lines derived from triple-negative breast cancer (TNBC) versus cell lines that were estrogen receptor (ER), progesterone receptor (PR) or human epidermal growth factor receptor 2 (HER2) positive.

### CDCA3 Levels Correlate with Response to Platinum Agents in TNBC

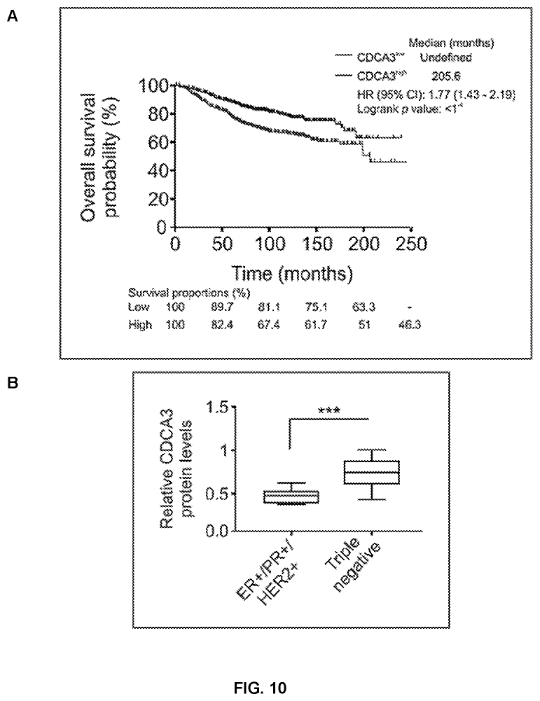

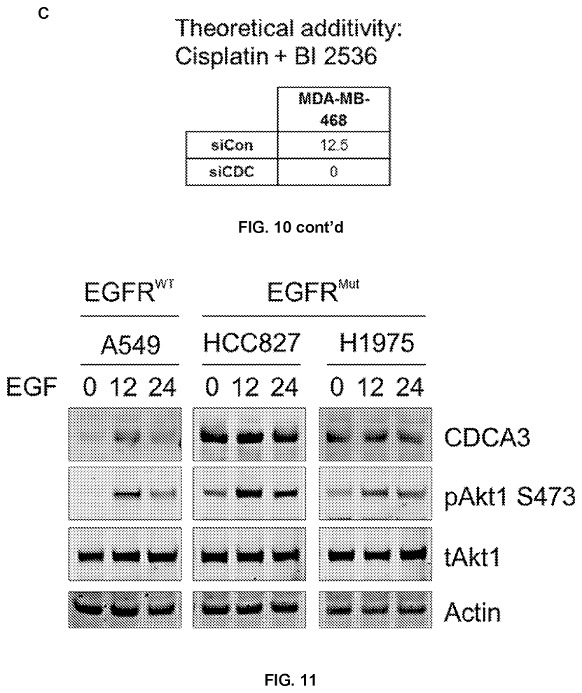

TNBC is the most aggressive subtype of breast cancer with chemotherapy the only available treatment. However, only 40-50% of TNBC patients have a partial response with the majority of patients relapsing with metastatic disease (Bianchini et al. 2016, Nature Reviews (https://www.nature.com/articles/nrclinonc.2016.66)). The present inventors tested whether cisplatin and the PLK1 inhibitor BI 2536 are additive in TNBC. Consistent with NSCLC cell lines, combination of cisplatin and BI 2536 resulted in 12.5% additive cell death in CDCA3 expressing cells versus no additivity in CDCA3 depleted cells (FIG. 10C).

### Discussion

The current data indicate that, as is the case in NSCLC, CDCA3 is prognostic in breast cancer and that TNBC cells with lower CDCA3 levels are more sensitive to platinum agents. Consistently, in breast cancer, use of PLK1 inhibitors may be most effective in tumour cells with elevated CDCA3, particularly in TNBC, which require novel treatment strategies to improve patient outcomes.

### EXAMPLE 3

The present Example further establishes CDCA3 expression as a prognostic indicator of therapeutic response to tyrosine kinase inhibition with or without PLK1 inhibition in NSCLC.

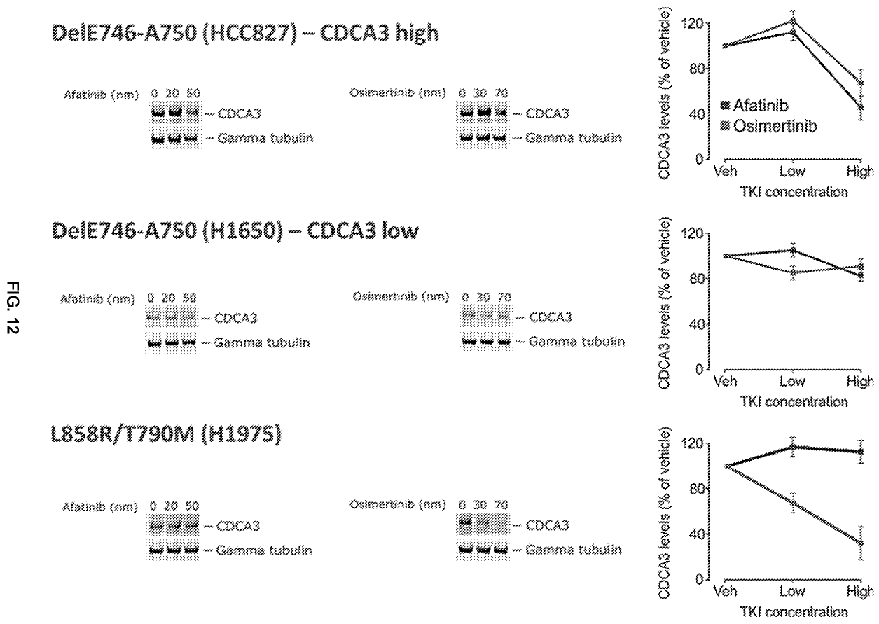

The current Example examined whether elevated CDCA3 was as a result of activating mutations in epidermal growth factor receptor (EGFR) that solely occur in cases of NSCLC adenocarcinoma. In in vitro models of disease, CDCA3 protein levels were examined by western blot analysis and it was identified that CDCA3 protein is markedly elevated in EGFR mutant NSCLC cell lines versus EGFR wildtype cell lines (FIG. 11). The present inventors also identified that stimulation of EGFR wildtype cells with EGF, an EGFR ligand, induced PI3K-Akt downstream signal transduction and the upregulation of CDCA3 protein. PI3K-Akt was constitutively activated in EGFR mutant NSCLC cell lines. These data suggest CDCA3 levels might be regulated downstream of a receptor tyrosine kinase.

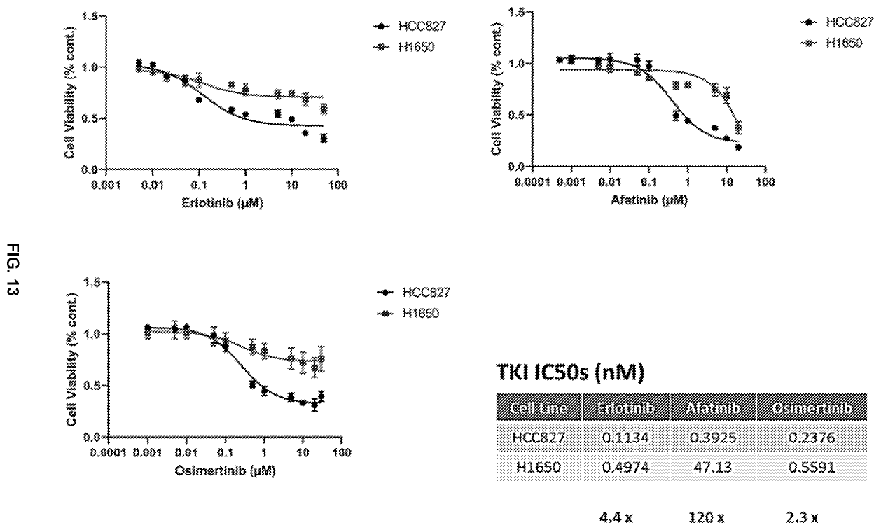

Tyrosine kinase inhibitors (TKI) are routinely employed upon clinical presentation of a case testing positive for activating mutations within EGFR. First (erlotinib, gefitinib), second (afatinib) and third generation (osimertinib) TKIs have been developed. While proving initially useful, tumour relapse remains a primary concern due to the prevalence of resistance mechanisms such as the acquisition of secondary EGFR mutations (e.g. T790M). Given CDCA3 levels are responsive to EGFR activation, the present inventors examined CDCA3 levels following second and third generation TKI treatment of three EGFR^{mut} cell lines; HCC827 and H1650 (exon 19 deletion E746-A750) and H1975 cells (L858R, T790M-resistant to first and second generation TKI). It was identified that CDCA3 levels varied with both HCC827 and H1975 exhibiting high CDCA3 whereas H1650 were CDCA3 low (FIG. 12). Upon TKI treatment, it was identified that CDCA3 levels were reduced in HCC827 cells in a dose dependent manner. In H1975 cells, CDCA3 levels were reduced only upon osimertinib treatment whereas CDCA3 levels were unaffected following TKI treatment in H1650 cells (FIG. 12). These data indicate that CDCA3 levels are indicative of how responsive EGFR mutant disease is to the appropriate TKI.

To evaluate the diagnostic potential for CDCA3 levels and TKI response, the present inventors performed dose response assays with TKIs in CDCA3 high (HCC827) versus low (H1650) EGFR mutant NSCLC cell lines. Both cell lines investigated were exon 19 del EGFR mutant. By assessing TKI potency and calculating drug IC₅₀ values, the cell viability data indicate that the CDCA3^{high} HCC827 cells versus CDCA3^{low} H1650 cells were 4.4 fold, 120 fold and 2.3 fold more sensitive to first, second and third generation TKIs respectively (FIG. 13). These data indicate the diagnostic potential for CDCA3 levels in EGFR mutant NSCLC.

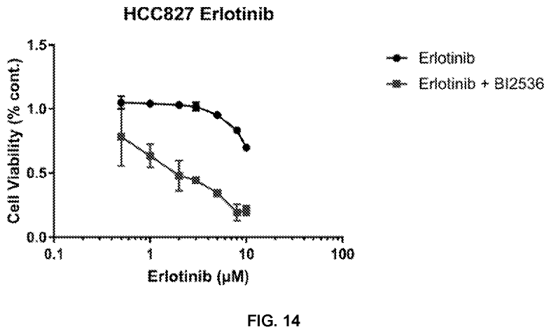

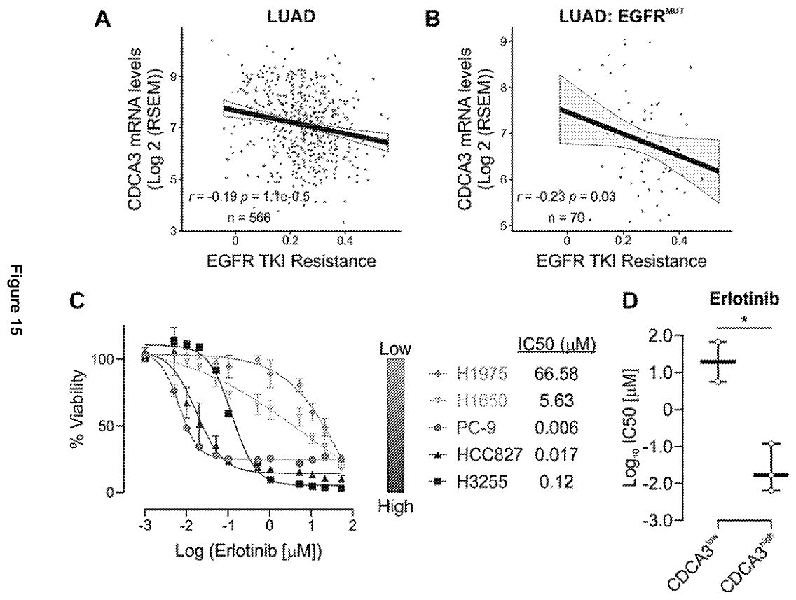

Given it was identified that PLK1 inhibitors are more potent in CDCA3^{high} NSCLC, the present inventors next evaluated whether PLK1 inhibition with BI 2536 is more effective when combined with TKI versus TKI alone. The current data indicate that when adding an IC₂₅ concentration of BI 2536 to escalating doses of erlotinib (first generation TKI), markedly reduced cell viability compared with TKI alone (FIG. 14). These data suggest that combining PLK1 inhibitors with TKIs can be a useful strategy to improve TKI sensitivity in CDCA3^{high} EGFR mutant NSCLC.

### EXAMPLE 4

### Materials and Methods

### Cell Culture, Transfections and Cell Treatments

All NSCLC cell lines were obtained from the American Type Culture Collection (ATCC) except for PC-9 cell line which was sourced from the European Collection of Cell Cultures (ECACC). Cells were grown in RPMI-1640-medium containing L-glutamine (Life Technologies) and 10% foetal bovine serum (FBS, Sigma Aldrich). HCC827 and PC-9 parental and erlotinib resistant cells generated by cyclic stepwise exposure to escalating doses of erlotinib (0.1 μM to 1 μM) over 6 months. Isogenic parental cell lines not exposed to erlotinib were maintained in culture over the same period. A549, H460 and H1299 cell lines are EGFR wildtype, whereas the H1650, HCC827, PC-9 cell lines have EGFR exon 19 deletions (E746_A750del), while H3255 cells are exon 21 EGFR mutant (L858R) and H1975 cells harbour both the L858R mutation and the T790M gatekeeper EGFR mutation. All cell lines were cultured at 37° C. in a humidified 5% CO₂ incubator and routinely tested for mycoplasma contamination. Transfection of CDCA3-FLAG expression construct was performed using the FuGene HD transfection reagent (Promega Corporation).

### Lysate Collection and Western Blot Analyses

To collect whole cell lysates, cells were first washed in PBS then lysed in lysis buffer (50 mM HEPES (pH 7.5), 150 mM KCl, 5 mM EDTA, 0.05% IGEPAL CA-630 (v/v), 1× protease inhibitor cocktail (Roche), and 1× phosphatase inhibitor cocktail (Cell Signalling Technology). Total protein was determined by bicinchoninic acid (BCA) protein assay (Sigma Aldrich) following lysate sonication and centrifugation. Samples (total protein 20 20 μg) were denatured in 1× Laemmli Buffer supplemented with 8% β-mercaptoethanol for 5 minutes at 80° C.

The samples were separated on Bolt 4-12% Bis-Tris Plus pre-cast gels (Invitrogen) and transferred onto nitrocellulose membrane (GE Healthcare Life Sciences) using the semi-dry Novex XCell II Blot Module transfer system (Life Technologies). The nitrocellulose membranes were blocked using Odyssey blocking buffer (Li-Cor) before incubation overnight at 4° C. with primary antibody in a 1:1 solution of PBS-T and Odyssey Blocking Buffer. Following primary antibody incubation, membranes were washed with PBS-T and incubated with the appropriate secondary antibodies. Membranes were scanned and imaged using the Li-Cor Odyssey (Li-Cor). Images were acquired and subject to densitometry analysis using the Image Studio Lite software.

### Cell Viability Assays

Cells were seeded into a white-walled, glass-bottomed 384-well plate (Nunc) at a density of 1x103 cells per well. The cells were treated with escalating doses of erlotinib, osimertinib or CX-4945 24 h following seeding over a period of 72 h. Cell viability was determined using CellTitre-Glo 2.0 (Promega Corporation) according to the manufacturer's instructions. Luminescence was scanned and analysed on the FLUOstar Omega Microplate Reader (BMG Labtech). Data was normalised to untreated controls and dose response curves and drug potency values generated using GraphPad Prism V9 software.

### Bioinformatics Analysis

CDCA3 mRNA expression levels were determined from TCGA RNA-seq datasets of EGFR wild-type LUAD NSCLC and EGFR-mutant LUAD NSCLC. CDCA3 expression levels were correlated against the publicly available WikiPathway “EGFR tyrosine kinase inhibitor resistance” parameter by linear regression analysis with P and R values calculated according to Spearman's rank correlation. Analyses were performed in the R statistical environment (R Core Team, Vienna, Austria). Cell line CDCA3 expression levels were determined from RNA-seq data accessed through cBioPortal.

### Results

### CDCA3 Correlates with Sensitivity to EGFR TKIs

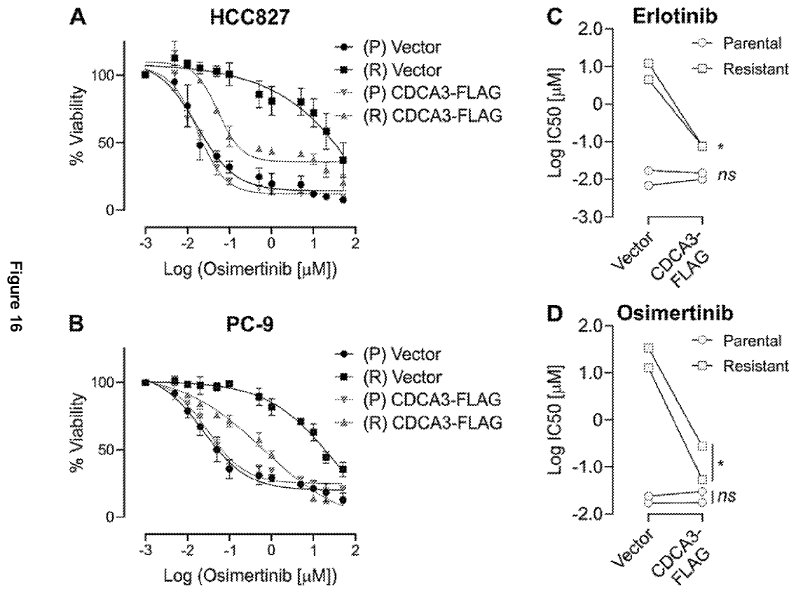

Given that the present inventors have previously demonstrated that CDCA3 strongly correlates with platinum-based chemotherapy response in all NSCLC histologies (1), it was next sought to determine whether a similar trend might exist with the response to EGFR TKIs. The present inventors evaluated TCGA datasets and correlated relative CDCA3 expression against available WikiPathways (2) of known measures of TKI resistance. In LUAD (FIG. 15A) and specifically in EGFR mutant LUAD (FIG. 15B), CDCA3 expression negatively correlated with resistance to EGFR TKI with respective Spearman correlation coefficients of R=−0.19 and R=−0.23. To experimentally confirm these clinical data correlations, the present inventors sought to investigate correlations between CDCA3 expression and in vitro TKI potency (IC₅₀) in a small panel of EGFR mutant NSCLC cell lines. Erlotinib (FIG. 15C) reduced cell viability in a dose-dependent manner. Consistently (3,4), H1975 cells, which harbour the T790M gatekeeper EGFR mutation, demonstrated resistance to erlotinib (IC₅₀=66.58 μM). Relative to other cell lines, H1650 cells were insensitive to erlotinib. Relative CDCA3 protein levels were correlated with the TKI potency values. Consistent with the bioinformatics analyses, CDCA3^{high} cell lines demonstrated greatest sensitivity to erlotinib (P=0.019, FIG. 15D).

### Upregulating CDCA3 Protein Levels in Models of Acquired EGFR TKI Resistance Enhances TKI Sensitivity

The present findings suggest strategies that upregulate CDCA3 might prove useful to enhance the sensitivity of EGFR mutant NSCLC to TKIs. The present inventors next sought to assess this possibility in in vitro models of acquired TKI resistance. To do so, TKI resistant models were generated in HCC827 and PC-9 cell lines by exposing cells to cycles of erlotinib over the course of 6 months. Cells not exposed to TKI were also maintained in culture for the same period, hereafter referred to as the parental cells. Although resistance was generated using erlotinib, these models exhibited marked osimertinib resistance with ˜385-fold and ˜1,470-fold increase in IC₅₀ values versus parental HCC827 (FIG. 16A) and PC-9 cell lines respectively (FIG. 16B). Although the precise mechanism of TKI resistance remains to be determined in these models, the emergence of T790M gatekeeper mutations in either cell line model was not detected (data not shown).

Having established two in vitro TKI acquired resistance models of EGFR mutant NSCLC, the present inventors next ectopically expressed CDCA3 in the parental and resistant pairs of each cell line. Ectopic expression of CDCA3 did not impact the sensitivity of HCC827 or PC-9 parental cells to erlotinib (FIG. 16C) or osimertinib (FIG. 16A,B,D). In contrast, ectopic CDCA3 expression significantly enhanced the potency of erlotinib (FIG. 16C) and osimertinib (FIG. 16A,B,D) by ˜79 and ˜121-fold respectively in HCC827 resistant cells. Similarly, ectopic CDCA3 expression significantly enhanced erlotinib and osimertinib potency by ˜57 and 54-fold respectively in PC-9 resistant cells.

### REFERENCES

1. Kildey, K., Gandhi, N. S., Sahin, K. B., Shah, E. T., Boittier, E., Duijf, P. H. G., Molloy, C., Burgess, J. T., Beard, S., Bolderson, E. et al. (2021) Elevating CDCA3 levels in non-small cell lung cancer enhances sensitivity to platinum-based chemotherapy. Communications Biology, 4, 638.
2. Martens, M., Ammar, A., Riutta, A., Waagmeester, A., Slenter, Denise N., Hanspers, K., A. Miller, R., Digles, D., Lopes, Elisson N., Ehrhart, F. et al. (2020) WikiPathways: connecting communities. Nucleic Acids Research, 49, D613-D621.
3. Mok, T. S., Wu, Y.-L., Ahn, M.-J., Garassino, M. C., Kim, H. R., Ramalingam, S. S., Shepherd, F. A., He, Y., Akamatsu, H., Theelen, W. S. M. E. et al. (2016) Osimertinib or Platinum-Pemetrexed in EGFR T790M-Positive Lung Cancer. New England Journal of Medicine, 376, 629-640.
4. Tang, Z.-H., Jiang, X.-M., Guo, X., Fong, C. M. V., Chen, X. and Lu, J.-J. (2016) Characterization of osimertinib (AZD9291)-resistant non-small cell lung cancer NCI-10 H1975/OSIR cell line. Oncotarget, 7, 81598-81610.

### REFERENCES

1. Global Burden of Disease Cancer C, Fitzmaurice C, Allen C et al. Global, Regional, and National Cancer Incidence, Mortality, Years of Life Lost, Years Lived With Disability, and Disability-Adjusted Life-years for 32 Cancer Groups, 1990 to 2015: A Systematic Analysis for the Global Burden of Disease Study. JAMA Oncol 2017; 3: 524-548.
2. Soerjomataram I, Lortet-Tieulent J, Parkin D M et al. Global burden of cancer in 2008: a systematic analysis of disability-adjusted life-years in 12 world regions. Lancet 2012; 380: 1840-1850.
3. Tone L A, Siegel R L, Jemal A. Lung Cancer Statistics. Adv Exp Med Biol 2016; 893: 1-19.
4. Australian Institute of H, Welfare. Cancer in Australia 2014: actual incidence data from 1982 to 2011 and mortality data from 1982 to 2012 with projections to 2014. Asia Pac J Clin Oncol 2015; 11: 208-220.
5. Melaku Y A, Appleton S L, Gill T K et al. Incidence, prevalence, mortality, disability-adjusted life years and risk factors of cancer in Australia and comparison with OECD countries, 1990-2015: findings from the Global Burden of Disease Study 2015. Cancer Epidemiol 2018; 52: 43-54.
6. Fennell D A, Summers Y, Cadranel J et al. Cisplatin in the modern era: The backbone of first-line chemotherapy for non-small cell lung cancer. Cancer Treat Rev 2016; 44: 42-50.
7. Ozkaya S, Findik S, Dirican A, Atici A G. Long-term survival rates of patients with stage IIIB and IV non-small cell lung cancer treated with cisplatin plus vinorelbine or gemcitabine. Exp Ther Med 2012; 4: 1035-1038.
8. Adams M N, Burgess J T, He Y et al. Expression of CDCA3 Is a Prognostic Biomarker and Potential Therapeutic Target in Non-Small Cell Lung Cancer. J Thorac Oncol 2017; 12: 1071-25 1084.
9. Gutteridge R E, Ndiaye M A, Liu X, Ahmad N. Plk1 Inhibitors in Cancer Therapy: From Laboratory to Clinics. Mol Cancer Ther 2016; 15: 1427-1435.
10. Lin C C, Su W C, Yen C J et al. A phase I study of two dosing schedules of volasertib (BI 6727), an intravenous polo-like kinase inhibitor, in patients with advanced solid malignancies. Br J Cancer 2014; 110: 2434-2440.
11. Schoffski P, Awada A, Dumez H et al. A phase I, dose-escalation study of the novel Polo-like kinase inhibitor volasertib (BI 6727) in patients with advanced solid tumours. Eur J Cancer 2012; 48: 179-186.
12. Stadler W M, Vaughn D J, Sonpavde G et al. An open-label, single-arm, phase 2 trial of the Polo-like kinase inhibitor volasertib (BI 6727) in patients with locally advanced or metastatic urothelial cancer. Cancer 2014; 120: 976-982.
13. Ellis P M, Leighl N B, Hirsh Vet al. A Randomized, Open-Label Phase II Trial of Volasertib as Monotherapy and in Combination With Standard-Dose Pemetrexed Compared With Pemetrexed Monotherapy in Second-Line Treatment for Non-Small-Cell Lung Cancer. Clin Lung Cancer 2015; 16: 457-465.
14. Lindqvist A, Rodriguez-Bravo V, Medema R H. The decision to enter mitosis: feedback and redundancy in the mitotic entry network. J Cell Biol 2009; 185: 193-202.
15. Matsuoka S, Ballif B A, Smogorzewska A et al. ATM and ATR substrate analysis reveals extensive protein networks responsive to DNA damage. Science 2007; 316: 1160-1166.
16. Russell P, Nurse P. Negative regulation of mitosis by wee 1+, a gene encoding a protein kinase homolog. Cell 1987; 49: 559-567.
17. Seki A, Coppinger J A, Jang C Y et al. Bora and the kinase Aurora a cooperatively activate the kinase Plk1 and control mitotic entry. Science 2008; 320: 1655-1658.
18. Gheghiani L, Loew D, Lombard B et al. PLK1 Activation in Late G2 Sets Up Commitment to Mitosis. Cell Rep 2017; 19: 2060-2073.
19. Hanahan D, Weinberg R A. Hallmarks of cancer: the next generation. Cell 2011; 144: 646-674.
20. Eastman A, Barry M A. Interaction of trans-diamminedichloroplatinum(II) with DNA: formation of monofunctional adducts and their reaction with glutathione. Biochemistry 1987; 26: 3303-3307.
21. Jamieson E R, Lippard S J. Structure, Recognition, and Processing of Cisplatin-DNA Adducts. Chem Rev 1999; 99: 2467-2498.
22. Koberle B, Tomicic M T, Usanova S, Kaina B. Cisplatin resistance: preclinical findings and clinical implications. Biochim Biophys Acta 2010; 1806: 172-182.
23. Sirzen F, Heiden T, Nilsson A et al. Characterisation of the G1/S cell cycle checkpoint defect in lung carcinoma cells with different intrinsic radiosensitivities. Anticancer Res 1997; 17: 3381-3386.
24. Smardova J, Liskova K, Ravcukova B et al. Complex analysis of the p53 tumor suppressor in lung carcinoma. Oncol Rep 2016; 35: 1859-1867.
25. Ayad N G, Rankin S, Murakami M et al. Tome-1, a trigger of mitotic entry, is degraded during G1 via the APC. Cell 2003; 113: 101-113.
26. Yoshida K. Cell-cycle-dependent regulation of the human and mouse Tome-1 promoters. FEBS letters 2005; 579: 1488-1492.
27. Itzel T, Scholz P, Maass T et al. Translating bioinformatics in oncology: guilt-by-profiling analysis and identification of KIF18B and CDCA3 as novel driver genes in carcinogenesis. Bioinformatics 2015; 31: 216-224.
28. Hu Q, Fu J, Luo B et al. OY-TES-1 may regulate the malignant behavior of liver cancer via NANOG, CD9, CCND2 and CDCA3: a bioinformatic analysis combine with RNAi and oligonucleotide microarray. Oncol Rep 2015; 33: 1965-1975.
29. Uchida F, Uzawa K, Kasamatsu A et al. Overexpression of cell cycle regulator CDCA3 promotes oral cancer progression by enhancing cell proliferation with prevention of G1 phase arrest. BMC cancer 2012; 12: 321.
30. Chen J, Zhu S, Jiang N et al. HoxB3 promotes prostate cancer cell progression by transactivating CDCA3. Cancer letters 2013; 330: 217-224.
31. Tang H, Xiao G, Behrens C et al. A 12-gene set predicts survival benefits from adjuvant chemotherapy in non-small cell lung cancer patients. Clin Cancer Res 2013; 19: 1577-1586.
32. Watanabe N, Arai H, Nishihara Y et al. M-phase kinases induce phospho-dependent ubiquitination of somatic Wee1 by SCFbeta-TrCP. Proc Natl Acad Sci U S A 2004; 101: 4419-4424.
33. Jaiswal H, Benada J, Mullers E et al. ATM/Wip1 activities at chromatin control Plk1 re-activation to determine G2 checkpoint duration. EMBO J 2017; 36: 2161-2176.
34. Eckerdt F, Yuan J, Strebhardt K. Polo-like kinases and oncogenesis. Oncogene 2005; 24: 267-276.
35. Sur S, Swier V J, Radwan M M, Agrawal D K. Increased Expression of Phosphorylated Polo-Like Kinase 1 and Histone in Bypass Vein Graft and Coronary Arteries following Angioplasty. PLoS One 2016; 11: e0147937.
36. Werle K, Chen J, Xu H G et al. Liver kinase B1 regulates the centrosome via PLK1. Cell Death Dis 2014; 5: e1157.
37. Yamanishi E, Hasegawa K, Fujita K et al. A novel form of necrosis, TRIAD, occurs in human Huntington's disease. Acta Neuropathol Commun 2017; 5: 19.
38. Ma X, Le Teuff G, Lacas B et al. Prognostic and Predictive Effect of TP53 Mutations in Patients with Non-Small Cell Lung Cancer from Adjuvant Cisplatin-Based Therapy Randomized Trials: A LACE-Bio Pooled Analysis. J Thorac Oncol 2016; 11: 850-861.
39. Pignon J P, Tribodet H, Scagliotti G V et al. Lung adjuvant cisplatin evaluation: a pooled analysis by the LACE Collaborative Group. J Clin Oncol 2008; 26: 3552-3559.
40. Karekla E, Liao W J, Sharp B et al. Ex Vivo Explant Cultures of Non-Small Cell Lung Carcinoma Enable Evaluation of Primary Tumor Responses to Anticancer Therapy. Cancer Res 2017; 77: 2029-2039.
41. Pearce N. Analysis of matched case-control studies. BMJ 2016; 352: i969.
42. Harrington B S, He Y, Davies C M et al. Cell line and patient-derived xenograft models reveal elevated CDCP1 as a target in high-grade serous ovarian cancer. Br J Cancer 2016; 114: 417-426.
43. He Y, Wu A C, Harrington BS et al. Elevated CDCP1 predicts poor patient outcome and mediates ovarian clear cell carcinoma by promoting tumor spheroid formation, cell migration and chemoresistance. Oncogene 2016; 35: 468-478.

1. A method of predicting the responsiveness of a cancer to treatment with a Bora-AurA-PLK1 pathway inhibitor in a subject, said method including the step of determining an expression level of a CDCA3 protein or encoding nucleic acid in one or a plurality of cancer cells, tissues or organs of the subject, wherein the expression level of the CDCA3 protein or encoding nucleic acid indicates or correlates with increased or decreased responsiveness of the cancer to treatment with the Bora-AurA-PLK1 pathway inhibitor in the subject.

2. The method of claim 1, wherein an increased level of the CDCA3 protein or encoding nucleic acid indicates or correlates with increased responsiveness of the cancer to the Bora-AurA-PLK1 pathway inhibitor; and/or a decreased level of CDCA3 protein or encoding nucleic acid indicates or correlates with decreased responsiveness of the cancer to the Bora-AurA-PLK1 pathway inhibitor.

3. The method of claim 1, wherein said method is for predicting the responsiveness of the cancer to treatment with the Bora-AurA-PLK1 pathway inhibitor and a further anti-cancer agent.

4. The method of claim 3, wherein an increased level of the CDCA3 protein or encoding nucleic acid indicates or correlates with increased responsiveness of the cancer to the Bora-AurA-PLK1 pathway inhibitor and the further anti-cancer agent; and/or a decreased level of CDCA3 protein or encoding nucleic acid indicates or correlates with decreased responsiveness of the cancer to the Bora-AurA-PLK1 pathway inhibitor and the further anti-cancer agent.

5-6. (canceled)

7. A method of treating cancer in a subject, the method including the step of determining an expression level of a CDCA3 protein or encoding nucleic acid in one or a plurality of cancer cells, tissues or organs of the subject and based on the determination made, initiating, continuing, modifying or discontinuing a cancer treatment in the subject, wherein the cancer treatment comprises administration of a Bora-AurA-PLK1 pathway inhibitor.

8. The method of claim 7, wherein the cancer treatment further comprises administration of a further anti-cancer agent.

9. The method of claim 7, further including the step of administering to the subject therapeutically effective amounts of the Bora-AurA-PLK1 pathway inhibitor and optionally a further anti-cancer agent when an increased level of CDCA3 protein or encoding nucleic acid is determined.

10. A method of treating cancer in a subject, said method including the step of administering a therapeutically effective amount of a Bora-AurA-PLK1 pathway inhibitor to the subject in which a level of CDCA3 protein or encoding nucleic acid has been determined in one or a plurality of cancer cells, tissues or organs of the subject that indicates or correlates with increased responsiveness of the cancer to the Bora-AurA-PLK1 pathway inhibitor.

11. The method of claim 10, wherein the method further includes administering a therapeutically effective amount of a further anti-cancer agent to the subject.

12-20. (canceled)

21. The method of claim 1, wherein the Bora-AurA-PLK1 pathway inhibitor is or comprises a PLK1 inhibitor.

22. The method of claim 3, wherein the further anti-cancer agent is or comprises a chemotherapeutic agent.

23-26. (canceled)

27. The method of claim 3, wherein the further anti-cancer agent is or comprises an EGFR inhibitor.

28-46. (canceled)

47. The method of claim 1, wherein the cancer is or comprises a lung cancer or a breast cancer, preferably wherein the lung cancer is or comprises NSCLC.

48-50. (canceled)

51. The method of claim 7, wherein the Bora-AurA-PLK1 pathway inhibitor is or comprises a PLK1 inhibitor.

52. The method of claim 8, wherein the further anti-cancer agent is or comprises a chemotherapeutic agent.

53. The method of claim 8, wherein the further anti-cancer agent is or comprises an EGFR inhibitor.

54. The method of claim 7, wherein the cancer is or comprises a lung cancer or a breast cancer, preferably wherein the lung cancer is or comprises NSCLC.

55. The method of claim 10, wherein the Bora-AurA-PLK1 pathway inhibitor is or comprises a PLK1 inhibitor.

56. The method of claim 11, wherein the further anti-cancer agent is or comprises a chemotherapeutic agent or an EGFR inhibitor.

57. The method of claim 10, wherein the cancer is or comprises a lung cancer or a breast cancer, preferably wherein the lung cancer is or comprises NSCLC.
